(19) United States
(12) Patent Application Publication
(10) Pub. No.: US 2013/0190845 A1
(43) Pub. Date: Jul. 25, 2013

(54) Edge-Lit Apparatus and Methods for Providing Light-Based Therapy

(71) Applicant: TRIA BEAUTY, INC., Dublin, CA (US)

(72) Inventors: Harvey I-Heng Liu, Fremont, CA (US); Patrick Reichert, Dublin, CA (US); David Youngquist, San Jose, CA (US); Tobin Island, Oakland, CA (US); Michael Patrick O'Neil, Dublin, CA (US); Mark V. Weckwerth, Pleasonton, CA (US)

(73) Assignee: TRIA BEAUTY, INC., Dublin, CA (US)

(21) Appl. No.: 13/750,103

(22) Filed: Jan. 25, 2013

Related U.S. Application Data
(60) Provisional application No. 61/590,559, filed on Jan. 25, 2012.
(60) Provisional application No. 61/645,751, filed on May 11, 2012.

(51) Int. Cl. A61N 5/06

(52) U.S. Cl. CPC A61N 5/0616
USPC 607/90

(57) ABSTRACT

An edge-lit device for providing light-based dermatological treatment is provided. The device may include a panel having internally reflective properties for reflecting light, the panel defining a front surface, a rear surface, a perimeter, and one or more edges extending along one or more portions of the perimeter. A rear reflector may be disposed adjacent the rear surface. The device may include one or more light emitting diodes (LEDs) positioned along at least one of the panel edges and configured to emit light into the panel such that at least a portion of the LED-emitted light reflects off the rear reflector and projects out of the panel through the panel front surface. The wavelength of the LED-emitted light may be suitable for dermatological treatment, such that LED-emitted light projecting out through the front surface of the panel may be useful for providing dermatological treatment to a user.

### CROSS-REFERENCE TO RELATED APPLICATIONS

This application claims the benefit of U.S. Provisional Application No. 61/590,559 filed on Jan. 25, 2012 and U.S. Provisional Application No. 61/645,751 filed on May 11, 2012; all of which applications are herein incorporated by reference in their entirety.

### TECHNICAL FIELD

The present disclosure is related to light-based therapy systems, e.g., edge-lit devices and methods for providing dermatological yellow light therapy.

### BACKGROUND

Low level or low-intensity light therapy has been clinically validated for treating a variety of dermatological conditions. For example, low-intensity yellow light therapy has been shown to treat dermatological conditions, such as, for example, wrinkles (e.g., periorbital wrinkles aka “crows feet”), superficial benign vascular lesions such as diffuse redness and telangiectasia or “spider veins”, or superficial benign pigmented lesions such as lentigos (age spots), solar lentigos (sun spots), or ephilides (freckles). One known product that has been developed to provide such low-intensity yellow light therapy includes an array of many LEDs positioned behind a transparent panel for emitting light through the panel and onto the treatment area of the user.

### SUMMARY

According to certain embodiments, an edge-lit device for providing light-based dermatological treatment is provided. The device may include a panel having internally reflective properties for reflecting light, the panel preferably defining a front surface, a rear surface, a perimeter, and one or more edges extending along at least one or more portions of the perimeter. A rear reflector may be disposed adjacent the rear surface. The device may include one or more light emitting diodes (LEDs) positioned along a panel edge and configured to emit light into the panel such that at least a portion of the LED-emitted light may reflect off the rear reflector and projects out of the panel through the panel front surface. The wavelength of the LED-emitted light may be suitable for dermatological treatment, such that LED-emitted light projecting out through the front surface of the panel may be used to provide dermatological treatment to a body part positioned in front of the front surface of the panel.

According to certain embodiments, an edge-lit device for providing light-based dermatological treatment is provided. The device may include a panel having internally reflective properties for reflecting light, the panel defining a front surface, a rear surface, a perimeter, and one or more edges extending along at least one or more portions of the perimeter. A rear reflector may be disposed adjacent the rear surface. The device may also include one or more light emitting diodes (LEDs) positioned along a panel edge and configured to emit light into the panel such that at least a portion of the LED-emitted light may reflect off the rear reflector and projects out of the panel through the panel front surface. The panel may be generally concave in at least one direction.

According to certain embodiments, an edge-lit device for providing light-based dermatological treatment is provided. The device may include a panel having internally reflective properties for reflecting light, the panel defining a front surface, a rear surface, a perimeter, and one or more edges extending along at least one or more portions of the perimeter. A rear reflector may be disposed adjacent the rear surface. The device may also include a plurality of light emitting diodes (LEDs) configured to emit light into the panel such that at least a portion of the LED-emitted light projects out through the panel front surface. The plurality of LEDs may include one or more first LEDs that emit light primarily at a first wavelength, and one or more second LEDs that emit light primarily at a second wavelength different than the first wavelength. At least one of the plurality of LEDs may be positioned along a panel edge and configured to emit light into the panel such that at least a portion of the LED-emitted light may reflect off the rear reflector and projects out of the panel through the panel front surface.

According to certain embodiments, an edge-lit device for providing light-based dermatological treatment is provided. The device may include a panel having internally reflective properties for reflecting light, the panel defining a front surface, a rear surface, a perimeter, and one or more edges extending along at least one or more portions of the perimeter. A rear reflector may be disposed adjacent the rear surface. The device may also include one or more light emitting diodes (LEDs) positioned along a panel edge and configured to emit light into the panel such that at least a portion of the LED-emitted light may reflect off the rear reflector and projects out of the panel through an illuminating area of the panel front surface. The ratio of (a) the number of LEDs used to illuminate the panel front surface to (b) the illuminating area of the panel front surface may be less than 1 LED per 10 cm̂2.

According to certain embodiments, a dermatological treatment device for a user is provided. The device may include one or more hand detectors, or one or more proximity sensors, and/or a controller. The controller may be configured to (a) receive hand detector signals from the one or more hand detectors; (b) determine, based on the received hand detector signals, whether the user is physically holding the dermatological treatment device; (c) receive proximity sensor signals from the one or more proximity sensors; (d) determine, based on the received proximity sensor signals, whether the user is positioned in a treatment position relative to the dermatological treatment device; and (e) automatically select between different states of operation based at least on the determinations of whether the user is physically holding the dermatological treatment device and whether the user is positioned in a treatment position relative to the dermatological treatment device.

According to certain embodiments, an edge-lit device for providing light-based dermatological treatment is provided. The device may include a first panel section and a second panel section coupled to each other, e.g., by a hinge. Each panel section may have internally reflective properties for reflecting light and defines a front surface, a rear surface, a perimeter, and one or more edges extending along at least one or more portions of the perimeter. Each panel section may include a rear reflector adjacent the rear surface. Each panel section may include one or more light emitting diodes (LEDs) positioned along a panel edge of that panel section and configured to emit light into that panel section such that at least a portion of the LED-emitted light may reflect off the rear reflector and projects out of that panel section through the panel front surface of that panel section.

Some embodiments provide a dermatological treatment device comprising: a panel having internally reflective properties for reflecting light, the panel defining a front surface, a rear surface, a perimeter, and one or more edges extending along one or more portions of the perimeter; a rear reflector adjacent the rear surface; and one or more light emitting diodes (LEDs) positioned along at least one of the panel edges and configured to emit light into the panel such that at least a portion of the LED-emitted light reflects off the rear reflector and projects out of the panel through the panel front surface; wherein a wavelength of the LED-emitted light is suitable for dermatological treatment, such that LED-emitted light projecting out through the front surface of the panel can be used to provide dermatological treatment to a body part positioned in front of the front surface of the panel.

In a further embodiment, the dermatological treatment device further comprises one or more brightness-enhancing layers positioned in front of the panel front surface. In a further embodiment, the dermatological treatment device further comprises one or more protective layers positioned in front of the panel front surface. In a further embodiment, the one or more LEDs are positioned outside the nearest panel edge. In a further embodiment, no LEDs are positioned behind the rear surface of the panel. In a further embodiment, the dermatological treatment device comprises multiple LEDs positioned along a particular panel edge. In a further embodiment, the dermatological treatment device comprises one or more LEDs positioned along a first panel edge; and one or more LEDs positioned along a second panel edge generally opposite or perpendicular to the first panel edge. In a further embodiment, the rear reflector comprises a diffuse reflector. In a further embodiment, the dermatological treatment device further comprises one or more edge reflectors extending along at least one panel edge. In a further embodiment, at least one edge reflector comprises a diffuse reflector. In a further embodiment, the one or more LEDs are positioned along one or more first panel edges; and the one or more edge reflectors are positioned along one or more second panel edges. In a further embodiment, the perimeter of the panel generally defines multiple sides; one or more LEDs are positioned along each of one or more of the multiple sides; and edge reflectors are positioned along each other one of the multiple sides. In a further embodiment, the panel is generally concave in at least one direction. In a further embodiment, the panel is generally concave about a first axis and also generally concave about a second axis perpendicular to the first axis. In a further embodiment, the degree of concavity about the first axis is greater than the degree of concavity about the second axis. In a further embodiment, at least one of the LEDs primarily emits yellow light at a wavelength of about 590 nm. In a further embodiment, the one or more LEDs include different LEDs that emit different wavelengths of light. In a further embodiment, the one or more LEDs include at least one LED that primarily emits yellow light at a wavelength of 590 nm+/−10 nm and at least one LED that primarily emits infrared light at a wavelength of 870 nm+/−20 nm. In a further embodiment, the one or more LEDs include at least one yellow light LED that primarily emits yellow light and at least one infrared LED that primarily emits infrared light. In a further embodiment, the one or more LEDs include more yellow light LEDs than infrared LEDs. In a further embodiment, at least one infrared LED is positioned between two yellow light LEDs along a side of the panel. In a further embodiment, the one or more LEDs include between 7 and 10 yellow light LEDs and between 5 and 10 infrared LEDs. In a further embodiment, the perimeter of the panel generally defines multiple sides; and different LEDs that emit different wavelengths of light are positioned along the same side of the panel. In a further embodiment, the panel is formed from acrylic. In a further embodiment, the panel comprises a bi-stable structure. In a further embodiment, the rear reflector includes one or more reflector scattering features that cause diffuse reflections of the LED-emitted light. In a further embodiment, at least one of the panel front surface and the panel rear surface includes internal reflection frustrating features that increase the external transmission of LED-emitted light from within the panel. In a further embodiment, the internal reflection frustrating features are distributed non-uniformly over the area of the panel.

In a further embodiment, the dermatological treatment device further comprises one or more light intensity uniformity features configured to alter the relative amounts of LED-emitted light projecting out of different areas of the panel front surface, such that the intensity of light projecting from the panel is generally uniform over the area of the panel front surface, the one or more light intensity uniformity features including at least one of: one or more reflector scattering features associated with the rear reflector; one or more internal reflection frustrating features associated with at least one of the panel front surface and the panel rear surface; and one or more curves in the panel. In a further embodiment, the one or more light intensity uniformity features provide a uniformity of light projection of +/−30% over the area of the panel front surface. In a further embodiment, the one or more light intensity uniformity features provide a uniformity of light projection of +/−20% over the area of the panel front surface. In a further embodiment, the dermatological treatment device further comprises one or more light intensity non-uniformity features configured to alter the relative amounts of LED-emitted light projecting out of different areas of the panel front surface, such that the intensity of light projecting through a first area of the panel front surface is greater than the intensity of light projecting through a second area of the panel front surface. In a further embodiment, the one or more light intensity non-uniformity features includes at least one of: one or more reflector scattering features associated with the rear reflector; one or more internal reflection frustrating features associated with at least one of the panel front surface and the panel rear surface; and one or more curves in the panel.

In a further embodiment, the dermatological treatment device further comprises a heat sink configured to remove heat from multiple LEDs. In a further embodiment, the dermatological treatment device further comprises one or more fans configured to cool the one or more LEDs. In a further embodiment, the dermatological treatment device further comprises a controller configured to operate the one or more LEDs in a pulsed manner. In a further embodiment, the controller is configured to pulse the one or more LEDs at a frequency of about 3 Hz. In a further embodiment, the controller is configured to pulse the one or more LEDs between “light” periods of about 250 ms and “dark” periods of about 100 ms. In a further embodiment, the dermatological treatment device further comprises one or more hand detectors configured to detect whether a user is physically holding the dermatological treatment device.

In a further embodiment, the dermatological treatment device further comprises a controller configured to automatically switch from a first operational state and a second operational state in response to the one or more hand detectors detecting that the user is physically holding the dermatological treatment device. In a further embodiment, the dermatological treatment device further comprises one or more proximity sensors configured to determine whether a user is positioned in a treatment position relative to the dermatological treatment device. In a further embodiment, the dermatological treatment device further comprises a controller configured to automatically switch from a first operational state and a second operational state in response to the one or more proximity sensors detecting that the user is positioned in a treatment position relative to the dermatological treatment device. In a further embodiment, the dermatological treatment device further comprises a controller configured to operate the one or more LEDs in a pulsed manner such that the LEDs alternate between “light” and “dark” periods; and wherein the proximity sensors are activated only during the “dark” periods of the one or more LEDs.

In a further embodiment, the panel comprises at least a first panel section and a second panel section coupled to each other; the one or more LEDs include: one or more first LEDs positioned to emit light through an edge of the first panel section; and one or more second LEDs positioned to emit light through an edge of the second panel section. In a further embodiment, the first and second panel sections are coupled to each other such that when the first and second panel sections are positioned in an open position, a first edge surface of the first panel section faces a second edge surface of the second panel section; and at least one of the first edge surface of the first panel section and the second edge surface of the second panel section is covered by an edge reflector. In a further embodiment, the first and second panel sections are coupled to each other such that when the first and second panel sections are positioned in an open position, a first edge surface of the first panel section faces a second edge surface of the second panel section; and the first edge surface and the second edge surface are optically active such that LED-emitted light flows between the first and second panel sections across the boundary between the first and second edge surfaces. In a further embodiment, the first and second panel sections are coupled to each other such that when the first and second panel sections are positioned in an open position, a first edge surface of the first panel section faces a second edge surface of the second panel section; and one or more connection ports or other user interfaces are formed in at least one of the first edge surface of the first panel section and the second edge surface of the second panel section.

In a further embodiment, during a treatment state of operation, a current of at least 100 mA is applied to each of the one or more LEDs. In a further embodiment, during a treatment state of operation, a power of at least 50 mW optical output is generated by each LED. In a further embodiment, the panel front surface has an illuminating area; and the ratio of (a) the number of LEDs used to illuminate the panel front surface to (b) the illuminating area of the panel front surface is less than 1 LED per 10 cm̂2. In a further embodiment, the panel front surface has an illuminating area; and the ratio of the number of LEDs used to illuminate the panel front surface to the illuminating area of the panel front surface is less than 1 LED per 25 cm̂2.

Another embodiment provides a dermatological treatment device, comprising: a panel having internally reflective properties for reflecting light, the panel defining a front surface, a rear surface, a perimeter, and one or more edges extending along one or more portions of the perimeter; a rear reflector adjacent the rear surface; and one or more light emitting diodes (LEDs) positioned along at least one of the panel edges and configured to emit light into the panel such that at least a portion of the LED-emitted light reflects off the rear reflector and projects out of the panel through the panel front surface; wherein the panel is generally concave in at least one direction.

In a further embodiment, the panel is generally concave about a first axis and also generally concave about a second axis perpendicular to the first axis. In a further embodiment, the degree of concavity about the first axis is greater than the degree of concavity about the second axis.

Another embodiment provides a dermatological treatment device, comprising: a panel having internally reflective properties for reflecting light, the panel defining a front surface, a rear surface, a perimeter, and one or more edges extending along one or more portions of the perimeter; a rear reflector adjacent the rear surface; and a plurality of light emitting diodes (LEDs) configured to emit light into the panel such that at least a portion of the LED-emitted light projects out through the panel front surface, including: one or more first LEDs that emit light primarily at a first wavelength; and one or more second LEDs that emit light primarily at a second wavelength different than the first wavelength; and wherein at least one of the plurality of LEDs is positioned along one the panel edges and configured to emit light into the panel such that at least a portion of the LED-emitted light reflects off the rear reflector and projects out of the panel through the panel front surface.

In a further embodiment, the one or more first LEDs primarily emit yellow light; and the one or more second LEDs primarily emit infrared light. In a further embodiment, the plurality of LEDs includes more yellow light LEDs than infrared LEDs. In a further embodiment, at least one infrared LED is positioned between two yellow light LEDs along a side of the panel. In a further embodiment, the plurality of LEDs includes between 7 and 10 yellow light LEDs and between 5 and 10 infrared LEDs. In a further embodiment, the perimeter of the panel generally defines multiple sides; and at least one yellow light LED and at least one infrared LED are positioned along the same side of the panel. In a further embodiment, the one or more first LEDs primarily emit yellow light at a wavelength of 590 nm+/−10 nm; and the one or more second LEDs primarily emit infrared light at a wavelength of 870 nm+/−20 nm.

Another embodiment provides a dermatological treatment device, comprising: a panel having internally reflective properties for reflecting light, the panel defining a front surface, a rear surface, a perimeter, and one or more edges extending along one or more portions of the perimeter, wherein the panel front surface has an illuminating area; a rear reflector adjacent the rear surface; and a plurality of light emitting diodes (LEDs) positioned along at least one of the panel edges and configured to emit light into the panel such that at least a portion of the LED-emitted light reflects off the rear reflector and projects out of the panel through the illuminating area of the panel front surface; wherein the ratio of (a) the number of LEDs used to illuminate the panel front surface to (b) the illuminating area of the panel front surface is less than 1 LED per 10 cm̂2.

Another embodiment provides a dermatological treatment device for a user, comprising: one or more hand detectors; and one or more proximity sensors; and a controller configured to: receive hand detector signals from the one or more hand detectors; determine, based on the received hand detector signals, whether the user is physically holding the dermatological treatment device, receive proximity sensor signals from the one or more proximity sensors; determine, based on the received proximity sensor signals, whether the user is positioned in a treatment position relative to the dermatological treatment device; and automatically select between different states of operation based at least on the determinations of whether the user is physically holding the dermatological treatment device and whether the user is positioned in a treatment position relative to the dermatological treatment device.

In a further embodiment, the controller is configured to: automatically switch from a first operational state to a second operational state in response to the one or more hand detectors detecting that the user is physically holding the dermatological treatment device; and automatically switch from the second operational state to the one or more proximity sensors detecting that the user is positioned in a treatment position relative to the dermatological treatment device. In a further embodiment, the first operational state is an “off” state; the second operational state is a “standby” state in which the one or more LEDs are activated to emit from the panel a ready state light intensity that is lower than a treatment state light intensity; and the third operational state is a “treatment” state in which the one or more LEDs are activated to emit the treatment state light intensity. In a further embodiment, the controller is further configured to automatically switch from the third operational state to the second operational state if the one or more hand detectors detect that the user is no longer physically holding the dermatological treatment device. In a further embodiment, the controller is further configured to automatically switch from the third operational state to the second operational state if the one or more proximity sensors detect that the user is no longer positioned in the treatment position relative to the dermatological treatment device.

In a further embodiment, the dermatological treatment device comprises a plurality of proximity sensors configured to take proximity readings at different locations on the user's body; and wherein the controller is configured to: determine, based on the received proximity sensor signals, multiple distances between the user and the dermatological treatment device; and compare the determined multiple distances to corresponding predetermined distance ranges to automatically determine whether the user is positioned in the treatment position relative to the dermatological treatment device. In a further embodiment, the plurality of proximity sensors includes a forehead proximity sensor and a chin proximity sensor.

Another embodiment provides a dermatological treatment device, comprising: a first panel section; a second panel section coupled to the first panel section; each panel section having internally reflective properties for reflecting light and having a front surface, a rear surface, a perimeter, and one or more edges extending along one or more portions of the perimeter; each panel section having a rear reflector adjacent the rear surface; and each panel section having one or more light emitting diodes (LEDs) positioned along at least one of the panel edges of that panel section and configured to emit light into that panel section such that at least a portion of the LED-emitted light reflects off the rear reflector and projects out of that panel section through the panel front surface of that panel section. In a further embodiment, the panel sections are coupled together by a hinge.

Another embodiment provides a dermatological treatment device, comprising: a panel having internally reflective properties for reflecting light, the panel defining a front surface, a rear surface, a perimeter, and one or more edges extending along one or more portions of the perimeter; a rear reflector adjacent the rear surface; and a plurality of light emitting diodes (LEDs) configured to emit light into the panel such that at least a portion of the LED-emitted light projects out through the panel front surface; wherein at least one of the LEDs emits both (a) yellow light at a first wavelength and (b) infrared light at a second wavelength different than the first wavelength; and wherein the at least one LED is positioned along at least one of the panel edges and configured to emit light into the panel such that at least a portion of the LED-emitted light reflects off the rear reflector and projects out of the panel through the panel front surface. In a further embodiment, at least one of the LEDs emits both (a) yellow light at a wavelength of about 590 nm and (b) infrared light at a wavelength of about 870 nm.

Another embodiment provides a method of treating skin using a dermatological treatment device comprising: aligning a treatment device with skin to be treated wherein the treatment device comprises a panel configured to emit light having internally reflective properties for reflecting light, the panel defining a front surface, a rear surface, a perimeter, and one or more edges extending along one or more portions of the perimeter; and causing the panel to emit light onto the skin with the following characteristics: a treatment time of approximately 15 seconds, a pulse rate of approximately 60 Hz, and a peak irradiance of 6 mW/cm²; wherein the emitted light includes light emitted at wavelengths within a band of about 521 nm to about 604 nm. In a further embodiment, the dermatological treatment device further comprises a reflective coating on the panel to at least partially reflect external light to allow use as a mirror.

### BRIEF DESCRIPTION OF THE DRAWINGS

Some embodiments of the disclosure may be understood by referring, in part, to the following description and the accompanying drawings, in which like reference numbers refer to the same or like parts and wherein:

FIG. 1 is a cross-sectional side view illustrating some components of an example device with an edge-lit LED panel for providing light-based dermatological treatment to a user, according to certain embodiments;

FIG. 2 is a front-view of the example edge-lit therapy device of FIG. 1, according to certain embodiments;

FIG. 3 is a front-view of another example edge-lit therapy device, including LEDs on multiple sides, according to certain embodiments;

FIG. 4 is a cross-sectional side view of an example edge-lit therapy device, showing a housing, according to certain embodiments;

FIG. 5 is a cross-sectional side view of an example light therapy device including LEDs positioned in a housing and within the outer perimeter of the panel, according to certain embodiments;

FIG. 6 is a front view of an example edge-lit therapy device, showing example heat dissipation components, according to certain embodiments;

FIG. 7 illustrates an example curved panel section of a dual-panel edge-lit therapy device, according to certain embodiments;

FIG. 8 is a top view of an example parabolic shape of the panels of a dual-panel edge-lit therapy device, according to certain embodiments;

FIG. 9 is a top view of an example semi-circular shape of the panels of a dual-panel edge-lit therapy device, according to certain embodiments;

FIG. 10 is a cross-sectional side view of an example edge-lit therapy device, showing various types and possible locations of sensors, according to certain embodiments;

FIG. 11 is a general block diagram of various operational components of an example light-based therapy device, according to certain embodiments;

FIG. 12 is a more detailed schematic of various operational components of a specific example of a light-based therapy device, according to certain embodiments;

FIG. 13 is a flow chart of an example method of operation for providing a treatment using a light-based therapy device, according to certain embodiments;

FIGS. 14A-14K illustrate various views of an example dual-panel edge-lit therapy device, according to certain embodiments;

FIGS. 15A-15H illustrate various views of another example dual-panel edge-lit therapy device, according to certain embodiments;

FIG. 16 illustrates top and front views of an example semi-circular panel for an edge-lit therapy device, showing example dimensions of the panel, according to certain embodiments;

FIG. 17 illustrates top and front views of an example parabolic panel for an edge-lit therapy device, showing example dimensions of the panel, according to certain embodiments;

FIGS. 18A-18C illustrate a treatment configuration and a storage configuration of an example edge-lit therapy device including panel sections coupled together and wherein the panel is concave around one axis, according to certain embodiments;

FIG. 19 illustrates partial components of an example edge-lit therapy device, in which batteries and LEDs are positioned along a vertical center section of the device, according to certain embodiments;

FIG. 20 illustrates an example display device for use with a light-based therapy device, according to certain embodiments;

FIGS. 21A-21C illustrate a first treatment configuration, a second treatment configuration, and a storage configuration, respectively, of an example edge-lit therapy device having folding panel, according to certain embodiments;

FIGS. 22A-22B illustrate a treatment configuration and a storage configuration, respectively, of an example edge-lit therapy device having a “compact-like” shape, according to certain embodiments;

FIGS. 23A-23B illustrate an example of a relatively-small edge-lit therapy device having a relatively small illuminating area, according to certain embodiments;

FIG. 24 illustrates an example edge-lit therapy device having a wand-like shape, according to certain embodiments;

FIG. 25 illustrates an example edge-lit therapy device configured similar to a vanity mirror, according to certain embodiments;

FIGS. 26A-26B illustrate an example edge-lit therapy device having an elongated, concave panel that is rotatably mounted to a base, according to certain embodiments; and

FIG. 27 illustrates an example waveform of a pulsed LED light treatment, according to certain embodiments.

### DETAILED DESCRIPTION

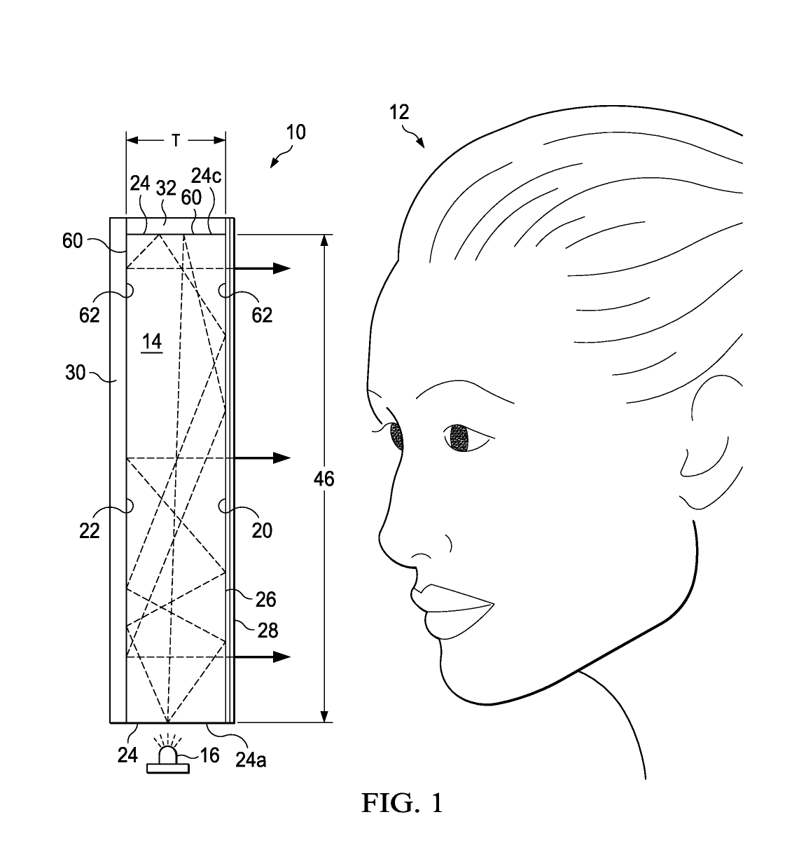

Selected embodiments of the disclosure may be understood by reference, in part, to FIGS. 1-27, wherein like numbers refer to same and like parts. The present disclosure relates generally to devices with edge-lit panels for providing low-intensity light therapy to users. For example, certain embodiments may be configured to provide low-intensity yellow therapy to a user's skin to treat one or more various dermatological conditions, such as, for example, wrinkles (e.g., periorbital wrinkles aka “crows feet”), superficial benign vascular lesions such as diffuse redness and telangiectasia or “spider veins”, or superficial benign pigmented lesions such as lentigos (age spots), solar lentigos (sun spots), or ephilides (freckles). Some embodiments may be particularly suitable for home use, thus replacing the need for professional treatments in a clinic. Thus, certain embodiments are easily hand-held and may provide full-face or partial-face coverage. In addition, such embodiments are generally portable and storable (e.g., in a vanity drawer or travel pouch or bag). In addition, the LED edge-lit design of certain embodiments may provide a substantially uniform illumination (or glow) over the area of the panel. Further, the LED edge-lit design may allow fewer LEDs to provide the desired treatment intensity, as compared to existing rear-lit designs that include a large array of LEDs behind a panel. Particular embodiments may benefit from one, some, or all of the advantages discussed herein. Further, various other advantages may be recognized by one of ordinary skill in the art.

For the purposes of this disclosure, an “edge-lit” panel is defined as an internally-reflective panel having at least one LED that emits light into the panel through (a) an edge surface of the panel or (b) through a non-edge surface (e.g., a front or rear surface) of the panel proximate an edge surface of the panel, and wherein the light emitted from the at least one LED into the panel generally reflects internally within the panel before projecting out through a front surface of the panel. Further, for the purposes of this disclosure, an edge-lit apparatus or device is defined as any apparatus or device having an edge-lit panel as defined above.

Thus, an “edge-lit” device or apparatus is distinguished from devices or apparatuses in which all LEDs are positioned behind a panel and configured to emit light directly through the panel, without internal reflection of the LED light within the panel.

### Example Panel Structure

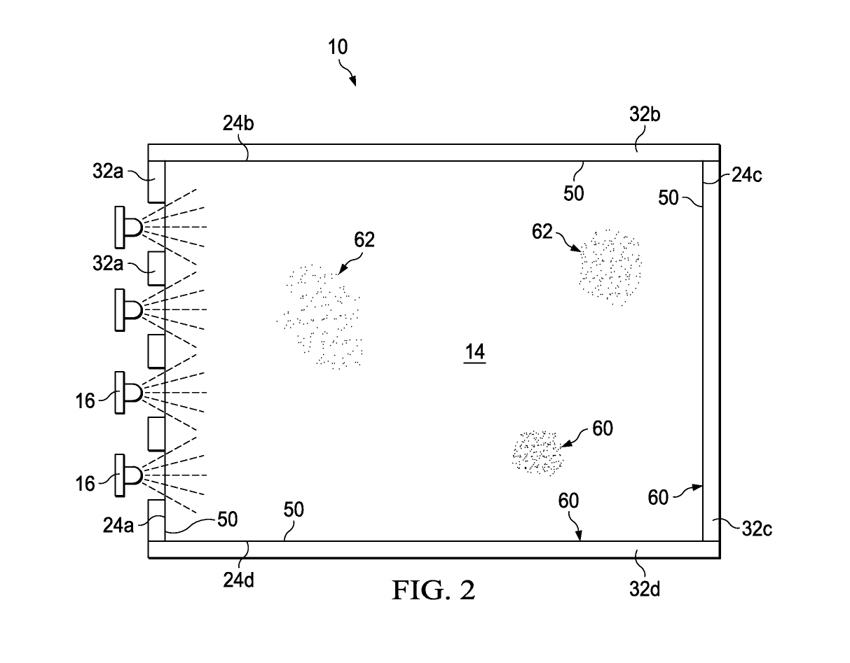

FIGS. 1 and 2 illustrate an example system including a dermatological treatment device 10 (hereafter referred to as “device 10”) for providing dermatological treatment to a user 12, according to certain embodiments of the present disclosure. In particular, FIG. 1 illustrates a cross-sectional side view of device 10, and FIG. 2 illustrates a front view of device 10.

Dermatological treatment device 10 is generally operable to apply low-intensity light treatment to the skin of user 12 to treat one or more various dermatological conditions. As shown in FIGS. 1 and 2, device 10 may include a panel 14 and one or more LEDs 16. Panel 14 may have a front surface 20, a rear surface 22, and one or more edges 24 (e.g., side edges 24a, 24b, etc. discussed below) extending along at least one or more portions of the perimeter of panel 14. In some embodiments, e.g., where panel 14 has a rectangular or generally rectangular shape, the perimeter may define a number of generally distinct sides such that panel 14 generally defines a number of side edges, such as side edges 24a, 24b, 24c, and 24d shown in FIGS. 1 and 2.

### Example Panel and Example Covering Layers

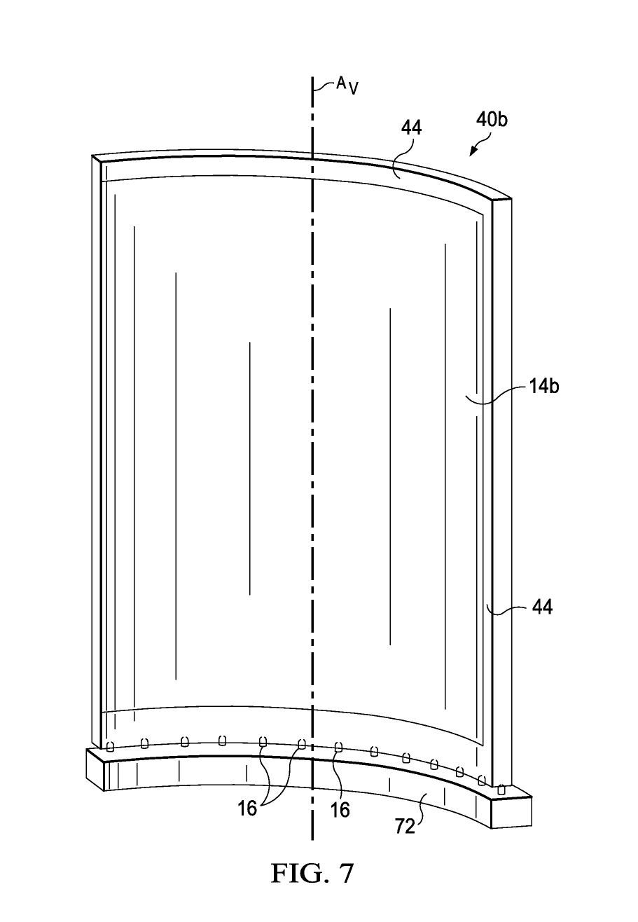

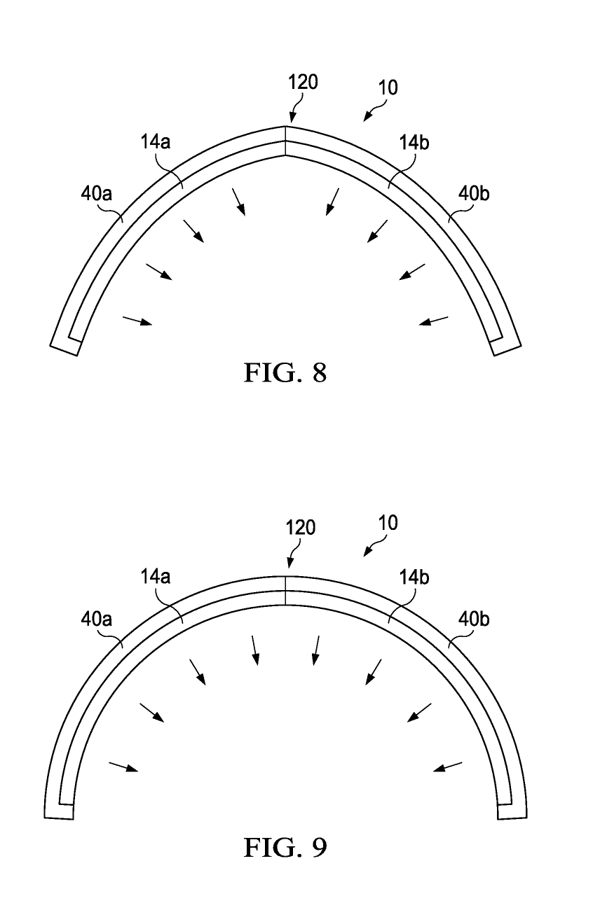

Panel 14 may be formed from any one or more materials having internally reflective properties for reflecting light, such that panel 14 acts as a light-guide or wave-guide. For example, panel 14 may be an acrylic panel. As other examples, panel 14 may be a polycarbonate panel, a suitably coated or enclosed glass panel, or chemically strengthened alkali-aluminosilicate sheet glass. Panel 14 may be planar or may include one or more curves, bends, or other non-planar contours. Some examples of planar panels 14 are shown, e.g., in FIGS. 1-6. Some examples of curved panels 14 are shown, e.g., in FIGS. 8, 9, 10, 12, 15, and 17. Device 10 may include a single panel 14 or multiple panel sections that cooperate in any suitable manner. For example, in the embodiments shown in FIGS. 8 and 9 (discussed below in greater detail), device 10 includes two panel sections 14a and 14b coupled together, in this instance by a hinged connection, such that device 10 may be folded between positions, e.g., between a storage position and a treatment position. In some embodiments, panel 14 (or panel sections) may be substantially planar. In other embodiments, panel 14 (or panel sections) may be curved or may include one or more curved portions. Curved panels or panel sections are discussed in greater detail below with reference to FIGS. 7-10, 14-19, 21, and 26.

In some embodiments, panel 14 may designed to be partially reflective to external light to allow the device to operate as a mirror when not operating in a light treatment mode. In some embodiments, the reflectivity of external light may be approximately 50%. For example, panel 14 could be incorporated into a bathroom mirror. In another example, panel 14 could be incorporated into a mirrored compact to allow discrete portability.

In some embodiments, one or more layers may be applied to the panel front surface 20. For example, one or more brightness-enhancing layers 26 and/or one or more protective layers 28 may be applied to panel 14. Brightness-enhancing layers 26 and/or protective layers 28 may comprise any suitable films, sheets, coatings, or other types of layers, and may be applied to panel 14 in any suitable manner (e.g., by mechanical fasteners, adhesive, heat treatment, chemical process, or by spraying or painting, for example). Brightness-enhancing layers 26 may, for example, be formed a prismatic film. Protective layers 28 may, for example, be formed from a thermoplastic polymer (e.g., polyethylene terephthalate).

### LED Positioning Along Panel Edge(s)

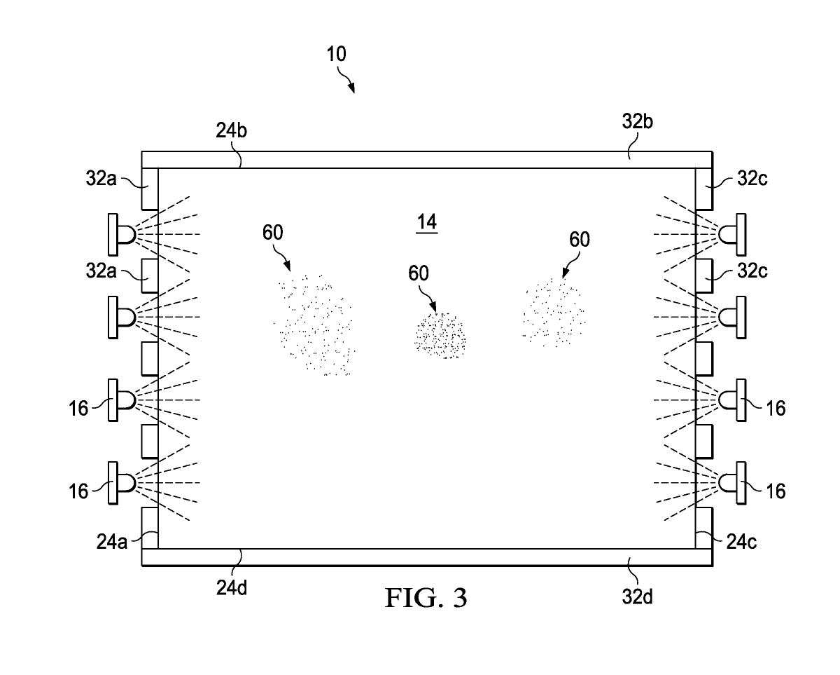

One or more LEDs 16 may be positioned along one or more portions of an edge, e.g., edge 24. For example, in the embodiment shown in FIG. 2, LEDs 16 may be positioned along side edge 24a. As another example, in the embodiment shown in FIG. 3, LEDs 16 may be positioned along opposite side edges 24a and 24c. In other embodiments, LEDs 16 may be positioned along any one, two, three, or all four side edges 24a, 24b, 24c, and 24d or portions thereof.

Any suitable number (zero, one, or more) of LEDs may be provided along each side edge 24a, 24b, 24c, and 24d. Some example embodiments include:
One LED 16 along only one side edge (e.g., side edge 24a).
One LED 16 along each of two opposite side edges (e.g., side edges 24a and 24c).
One LED 16 along each of two adjacent side edges (e.g., side edges 24a and 24b).
One LED 16 along each of three side edges (e.g., side edges 24a, 24b, and 24c).
One LED 16 along on all side edges (e.g., side edges 24a, 24b, 24c, and 24d).
Multiple LEDs 16 along only one side edge (e.g., side edge 24a).
Multiple LEDs 16 along each of two opposite side edges (e.g., side edges 24a and 24c).
Multiple LEDs 16 along each of two adjacent side edges (e.g., side edges 24a and 24b).
Multiple LEDs 16 along each of three side edges (e.g., side edges 24a, 24b, and 24c).
Multiple LEDs 16 along on all side edges (e.g., side edges 24a, 24b, 24c, and 24d).

In embodiments with multiple LEDs along more than one side edge, the same number or different numbers of LEDs may be provided along the different side edges.

In embodiments in which the perimeter generally does not have defined sides, e.g., a circular or oval shaped panel 14, any suitable number of LEDs 16 may be positioned along one or more portions of the perimeter edge 24, or along the entire perimeter edge 24.

LEDs 16 are positioned along edge 24 such that light emitted from the LEDs enters into panel 14 through edge 24, is internally reflected any number of times within the thickness of panel 14, and projects out through one or more surfaces of panel 14.

### Reflectors

In some embodiments, device 10 includes one or more reflectors for directing the LED-emitted light out through one or more particular surfaces of panel 14. For example, device 10 may include reflectors for directing the LED-emitted light out through only the front surface 20 of panel 14, or through only the front surface 20 and one or more particular side edges 24a-24d of panel 14. Thus, device 10 may include a rear panel reflector 30 covering the panel rear surface 22, and one or more edge reflectors 32 covering one or more portions of panel edge 24. One, some, or all reflectors 30 and 32 may comprise diffuse reflectors formed from any suitable diffuse reflective material(s), such as white plastic or a reflective oxide layer (e.g., Al₂O₃ or TiO₂), for example. Reflectors 30 and 32 may have any suitable structure (e.g., rigid or semi-rigid panels, thin metallic films, or coatings, for example) and may be coupled, bonded, or applied to the surfaces of panel 14 in any suitable manner (e.g., by mechanical fasteners, adhesive, heat treatment, chemical process, or by spraying or painting, for example).

In the embodiment shown in FIGS. 1 and 2, device 10 includes a rear panel reflector 30 covering the panel rear surface 22, and edge reflectors 32b, 32c, and 32d covering side edges 24b, 24c, and 24d, respectively, as well as edge reflectors 32a covering portions of side edge 24a between the LEDs 16. Thus, in this embodiment, reflectors 30 and 32 are designed to direct as much of the LED-emitted light as possible out through the front surface 20 of panel 14.

### Housing

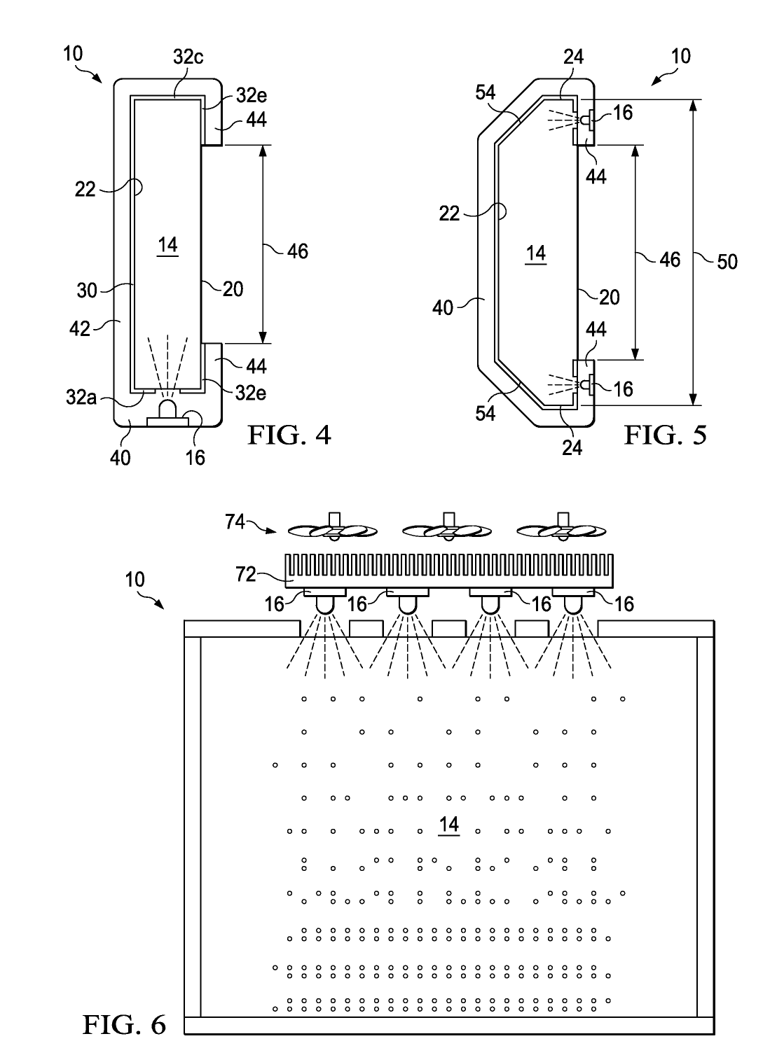

Device 10 may include a frame or housing and/or a bezel. For example, FIG. 4 illustrates a cross-sectional side view of an example device 10 including a housing 40 for housing panel 14, LEDs 16, and reflectors 30 and 32. Housing 40 may include a rear panel 42 and/or a bezel 44 that covers at least a portion of the front surface 20 of panel 14. Bezel 44 may wrap around any one, two, three, or all sides of panel 14. Device 10 may further include bezel reflectors 32e covering portions of front surface 20 covered by bezel 44. In such embodiments, an illuminating area 46 of the front surface 20 is defined as the area of the front surface 20 of panel 14 not covered by bezel 44 or any other structure (i.e., the area through which light can project toward the user). In embodiments without a bezel and/or any other structures covering any portion of the front surface 20 of panel 14, the illuminating area 46 is equal to the area of the front surface 20.

Reflectors 30 and/or 32 may be coupled, bonded, or applied to the outer surfaces of panel 14 and/or to the inner surfaces of housing 40. Alternatively, reflectors 30 and/or 32 may be formed integral with housing 40. For example, reflectors 30 and/or 32 may simply comprise the inner surfaces of housing 40. Panel 14 may be coupled to or mounted in housing 40 in any suitable manner, such as by mechanical fasteners, adhesive, heat treatment, or chemical process, for example.

### LED Positioning for Providing an “Edge-Lit” Panel

In some embodiments, all LEDs 16 are positioned outside the illuminating area 46 of the front surface 20 and proximate the perimeter 50 defined by panel edge(s) 24. In these embodiments, no LEDs 16 are positioned behind the rear surface 22 of panel 14. For example, in the embodiments shown in FIGS. 1-4, all LEDs 16 are positioned outside the perimeter 50 defined by panel edge(s) 24. LEDs 16 may be centered relative to the thickness T (see FIG. 1) of panel 14. Alternatively, LEDs 16 may be offset relative to the center of thickness T. Further, LEDs 16 may be spaced apart from each other along one or more side edges 24a-24d in any suitable manner. For example, LEDs 16 may be uniformly spaced apart from each other along one or more side edges 24a-24d. Alternatively, LEDs 16 may be spaced apart from each other in a non-uniform, but symmetric pattern along one or more side edges 24a-24d. Alternatively, LEDs 16 may be spaced apart from each other in a non-uniform and non-symmetric pattern along one or more side edges 24a-24d. In some embodiments, the configuration (e.g., the number, location, spacing, orientation, etc.) of LEDs 16 is selected to provide a generally uniform or intentionally non-uniform (depending on the embodiment) intensity of light over the illuminating area 46 of panel 14. For example, in some embodiments, the configuration (e.g., the number, location, spacing, orientation, etc.) of LEDs 16 is designed to provide a light output uniformity of +/−30% over the entire (or substantially entire) illuminating area 46. In particular embodiments, the configuration (e.g., the number, location, spacing, orientation, etc.) of LEDs 16 is designed to provide a light output uniformity of +/−20% over the entire (or substantially entire) illuminating area 46.

As another example, e.g., as shown in the embodiment in FIG. 5, LEDs 16 may be positioned within the perimeter 50 defined by panel edge(s) 24, but outside the illuminating area 46 and proximate panel edge(s) 24. In this example, LEDs 16 are positioned within or behind bezel 44 and emit light toward the rear surface 22 of panel 14, which may include curved or angled portions 54 designed for distributing the light as desired.

Other embodiments include one or more LEDs 16 positioned outside the illuminating area 46 of the front surface 20, and one or more LEDs 16 positioned within the illuminating area 46 of the front surface 20. For example, one embodiment includes one or more first LEDs 16 positioned outside a perimeter edge 24 of panel 14 and emit light into panel 14 through the edge 24 (as discussed above with reference to FIGS. 1-4), and one or more second LEDs 16 positioned behind panel 14 (within the illuminating area 46) and emit light into panel 14 through rear surface 22. In such embodiments, the first and second LEDs may be activated simultaneously (e.g., either as supplementary parts of the same type of treatment or for different types of treatments), or alternatively, may be activated separately (e.g., for different types of treatments).

### Types of LEDs

LEDs 16 may include one type of LED or multiple different types of LEDs. In some embodiments, all LEDs 16 are yellow light LEDs that primarily emit yellow light. For example, LEDs 16 may primarily emit yellow light at a wavelength of about 590 nm+/−20 nm. In some embodiments, some or all yellow light LEDs 16 may simultaneously emit a secondary infrared emission. For example, in some embodiments, some or all yellow light LEDs 16 emitting 590 nm wavelength yellow light may simultaneously emit a secondary infrared emission with a wavelength of about 870 nm. The secondary infrared emission of such yellow light LEDs may provide one or more photobiological effects, e.g., for treating dermatological condition(s), tanning, aiding in vitamin D synthesis, reducing the effects of Seasonal Affective Disorder (SAD), or for providing any other benefits to the user's health or appearance.

Embodiments including yellow light LEDs 16 may include any suitable number of yellow light LEDs to provide the desired intensity of yellow light delivered to the user. For example, in certain embodiments, device 10 may include a suitable number of yellow light LEDs to provide a generally uniform yellow light intensity over the illuminating area 46 of about 4 mW/cm².

In other embodiments, LEDs 16 may include one or more yellow light LEDs that primarily emit yellow light and one or more LEDs that primarily emit a different wavelength of visible or non-visible light. For example, LEDs 16 may include one or more yellow light LEDs that primarily emit yellow light and one or more infrared LEDs that primarily emit infrared light. For instance, in some embodiments, a device 10 may include one or more yellow light LEDs that primarily emit yellow light at a wavelength of 590 nm+/−10 nm and one or more infrared LEDs that primarily emit infrared light at a wavelength of 870 nm+/−20 nm.

In embodiments that include both yellow light LEDs and non-yellow light LEDs (e.g., infrared LEDs), the different LEDs may be arranged in any suitable manner relative to panel 14 and relative to each other. For example, device 10 may include yellow light LEDs arranged along one or more side edges 24a-24d of panel 14, and infrared LEDs arranged along one or more other side edges 24a-24d of panel 14. As another example, yellow light LEDs and infrared LEDs may be arranged along the same side edge 24a-24d, with the different LED types grouped together or intermixed with each other. One or more infrared LEDs may be arranged in line between two yellow light LEDs, and vice versa. For example, yellow light LEDs and infrared LEDs may be arranged in an alternating manner, in a regular or irregular pattern.

LEDs 16 may be spaced apart from each other along one or more side edges 24a-24d in any suitable manner, regardless of whether device 10 includes one or multiple types of LEDs. For example, as discussed above, LEDs 16 may be uniformly spaced apart from each other, or non-uniformly but symmetrically spaced apart from each other, or non-uniformly and non-symmetrically spaced apart from each other, along one or more side edges 24a-24d.

As discussed above, in some embodiments, the configuration (e.g., the number, location, spacing, orientation, etc.) of LEDs 16 is designed to provide a light output uniformity of +/−30% over the entire (or substantially entire) illuminating area 46. In particular embodiments, the configuration (e.g., the number, location, spacing, orientation, etc.) of LEDs 16 is designed to provide a light output uniformity of +/−20% over the entire (or substantially entire) illuminating area 46.

In some embodiments, LEDs 16 may include one or more Philips Luxeon Rebel Phospher Converted Amber LEDs (for yellow light or primarily yellow light emissions) and one or more Light-on HSDL-44xx IR Emitters (for infrared light or primarily infrared light emissions). In one example embodiment, device 10 includes two panel sections 14a and 14b, and for lighting each panel section, eight Philips Luxeon Rebel Phospher Converted Amber LEDs and eight Light-on HSDL-44xx IR Emitters.

### Scattering and Reflection Frustration Features

Device 10 may include any one or more types of intensity control features designed to provide a desired pattern of light intensity emitted by panel 14 by influencing reflection of light emitted by LEDs 16, e.g., to increase the total light intensity emitted from panel 14, to provide a generally uniform intensity of emitted light over the illuminating area 46 of panel 14, or to provide an intentionally non-uniform intensity of emitted light from panel 14 over the illuminating area 46.

Types of intensity control features include, for example, (a) scattering or diffusive features 60 associated with reflectors 30 and/or 32, (b) internal reflection frustrating features 62 associated with the panel front surface 20 and/or the panel rear surface 22, and (c) curves or other non-planar contours in panel 14. Device 10 may include any one or more of these features in any combination. For example, FIG. 1 shows scattering or diffusive features 60 on both rear reflector 30 and side reflector 32, and internal reflection frustrating features 62 on both panel front surface 20 and panel rear surface 22. However, depending on the embodiment, features 60 may or may not be provided on particular reflectors 30 and/or 32, and features 62 may or may not be provided on particular surfaces 22 and/or 22, in any suitable combination. For example, in one embodiment, features 60 are provided on all reflectors 30 and 32, and features 62 are provided on panel rear surface 22 but not on panel front surface 20.

Scattering or diffusive features 60 associated with reflectors 30 and/or 32 may include any features that increase or decrease the diffusiveness of reflectors 30 and/or 32, e.g., surface abrasions or roughness, or granular coatings.

Internal reflection frustrating features 62 include any features on panel front surface 20 and/or the panel rear surface 22 that cause light to transmit through the respective surface 20 or 22 rather than reflect internally (within panel 14) off that surface 20 or 22. Specifically, light which strikes the relevant surface 20 or 22 at an angle larger than the relevant critical angle with respect to the normal to the surface 20 or 22 is redirected by features 62 through the respective surface 20 or 22 (and out of panel 14), rather than reflecting internally (within panel 14) off the respective surface 20 or 22. Thus, such features are said to “frustrate” (i.e., reduce) the internal reflection of light within panel 14. Internal reflection frustrating features 62 may include, e.g., surface abrasions, roughness, cuts, or any other non-planar features in surface 20 or 22. In some embodiments, panel 14 includes internal reflection frustrating features 62 on panel rear surface 22 such that LED-emitted light strikes rear surface 22, is transmitted out through rear surface 22, then reflects off rear reflector 30 and through panel 14 and toward the user. Thus, adding internal reflection frustrating features 62 to a particular area of panel 14 generally increases the emission intensity from that area of panel 14, relative to other areas. Further, the greater the concentration of internal reflection frustrating features 62 in a particular area of panel 14, the greater the emission intensity from that area of panel 14, relative to other areas. Internal reflection frustrating features 62 may be used separate from, or in combination with, scattering or diffusive features 60 on reflectors 30 and/or 32.

Any of intensity control features discussed above may be used either for providing a generally uniform intensity of emitted light over illuminating area 46 or for providing an intentionally non-uniform intensity of emitted light from panel 14 over the illuminating area 46, depending on how the particular features are oriented or distributed throughout the device. For example, to provide a generally uniform intensity of emitted light over illuminating area 46, internal reflection frustrating features 62 may be provided in a predetermined pattern with increasing concentration as a function of increasing distance from LEDs 16, e.g., as illustrated in FIG. 2. In some embodiments, features 60 and/or 62 may help provide a light output uniformity of +/−30% over the entire (or substantially entire) illuminating area 46. In particular embodiments, features 60 and/or 62 may help provide a light output uniformity of +/−20% over the entire (or substantially entire) illuminating area 46.

As discussed above, in some embodiments, the configuration (e.g., the number, location, spacing, orientation, etc.) of LEDs is designed to provide a light output uniformity of +/−30%, or even +/−20%, over the entire (or substantially entire) illuminating area 46. In such embodiments, features 60 and/or 62 may further increase the light output uniformity over illuminating area 46.

As another example, internal reflection frustrating features 62 may be provided in a predetermined pattern of varying concentrations such that one or more selected regions (e.g., the top, bottom, left, right, and/or center) of illuminating area 46 emit light at a higher intensity than other regions. Such patterns may be used, for example, to provide a non-uniform intensity profile designed to correspond to non-planar or irregular contours of a body part to be treated by device 10. For instance, a device 10 having a planar panel 14 intended for treating a three-dimensionally curved body part (e.g., the head or thigh) may include a pattern of features 62 designed to provide higher emission intensity from the outer portions of illuminating area 46 (as compared to the center of area 46), in order to compensate for the greater distance between the outer portions of illuminating area 46 and the treated body part (as compared to the distance between the center of area 46 and the treated body part). In this manner, a non-uniform light emission intensity over area 46 can deliver a relatively uniform light intensity to the treatment area.

In some embodiments, illustrated in FIG. 6, the arrangement of features 60 and/or 62 (indicated as dots on panel 14) are arranged in a sparse pattern closest to LEDs 16. This arrangement becomes progressively less sparse as the distance from the LEDs to the features increases until a solid pattern is maintained at the furthest region of panel 14. This progressive pattern provides a high degree of uniformity in light output in the following manner. The intensity of the light produced by LEDs 14 diminishes as a function of distance from each LED. In the region closest to the LEDs, a small number of sparse features 60 and/or 62 will result in a given light output. At a further distance, the light intensity will be reduced, due to natural dissipation and/or absorption by the substrate forming panel 14. To achieve the same level of output, a higher density of features 60 and/or 62 is required. In some embodiments, features 60 and/or 62 may be arranged in a regular grid with more gaps in the area nearest LEDs 16 and a number of rows, six in one embodiment, in the area of panel 14 furthest from LEDs 16. In still other embodiments, features 60 and/or 62 may be arranged in random or approximately random patterns with progressive density from sparse to dense in the manner described above.

### Heat Dissipation

Device 10 may include any suitable passive and/or active heat control systems for controlling heat generated by any heat-generating components of device 10 (e.g., LEDs 16 and/or other electronic components). Such heat control systems may be configured, for example, to dissipate heat (e.g., to spread heat generally evenly across device 10 or an area of device 10, or to transfer heat toward an outer surface for external dissipation) or to move heat away from particular areas of device 10 (e.g., generally away from the user's hands).

In some embodiments, device 10 includes a fully passive heat control system including one or more thermally conductive elements (e.g., one or more heat sinks or other conductive elements) physically coupled to one or more heat-generating components of device 10. One or more thermally conductive elements may extend along the outer surfaces of device 10 or within the thickness of device 10. In some embodiments, one or more portions of housing 40, or other outer surfaces of device 10, may be formed from a thermally conductive material, in order to dissipate or transfer the heat across device 10 as desired.

Alternatively, or in addition to such passive heat control systems, device 10 may include any suitable active heat control systems, such as one or more powered fans, for example. In the example embodiment shown in FIG. 6, device 10 includes a heat control system 70 including a heat sink 72 and one or more fans 74. A number of LEDs 16 and/or other electronics are physically mounted on heat sink 72 to dissipate heat away from such components. Heat sink 72 may have any suitable size and/or shape, may be formed from any suitable material or materials (e.g., aluminum or other thermally conductive material or materials), and may include fins or any other known heat dissipation or heat transfer features. Fans 74 may be arranged to pass air over heat sink 72 to provide or increase convective heat transfer. Heat sink 72 and one or more fans 74 may be disposed within housing 40, which may include any suitable slots, holes, or other openings configured to promote air flow for convective cooling.

In one example embodiment, 16 LEDs are arranged on a passive aluminum heat sink 72 having dimensions of about 0.6 cm thick, 1.1 cm wide, and 12 cm long.

### Curved Panels/Half-Panels

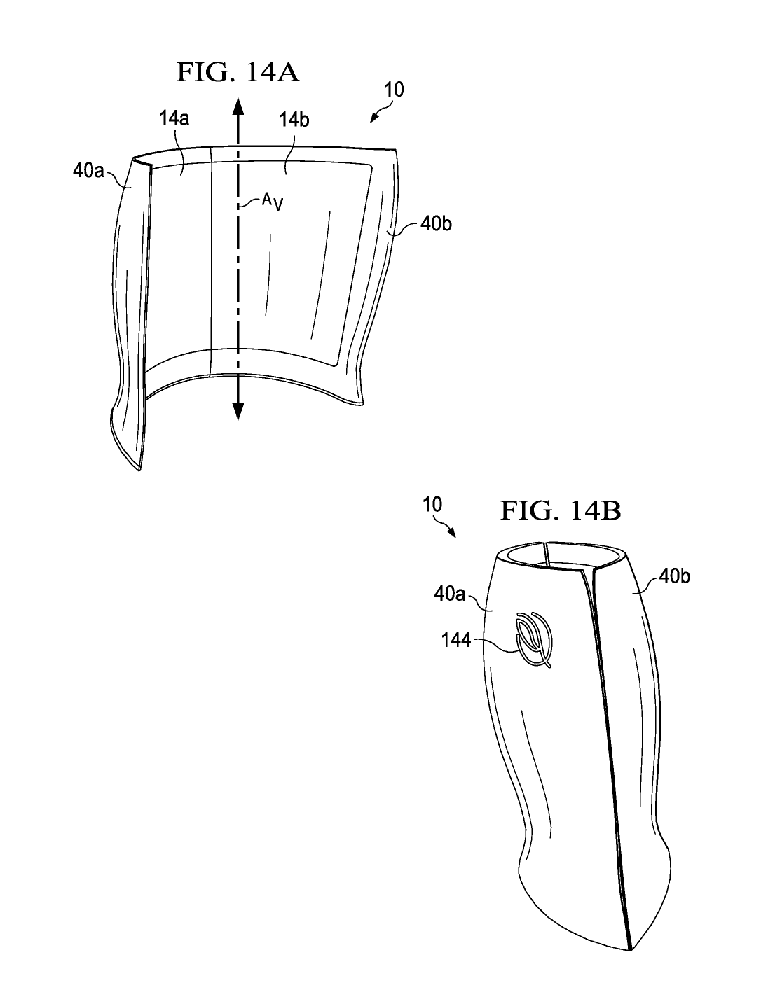

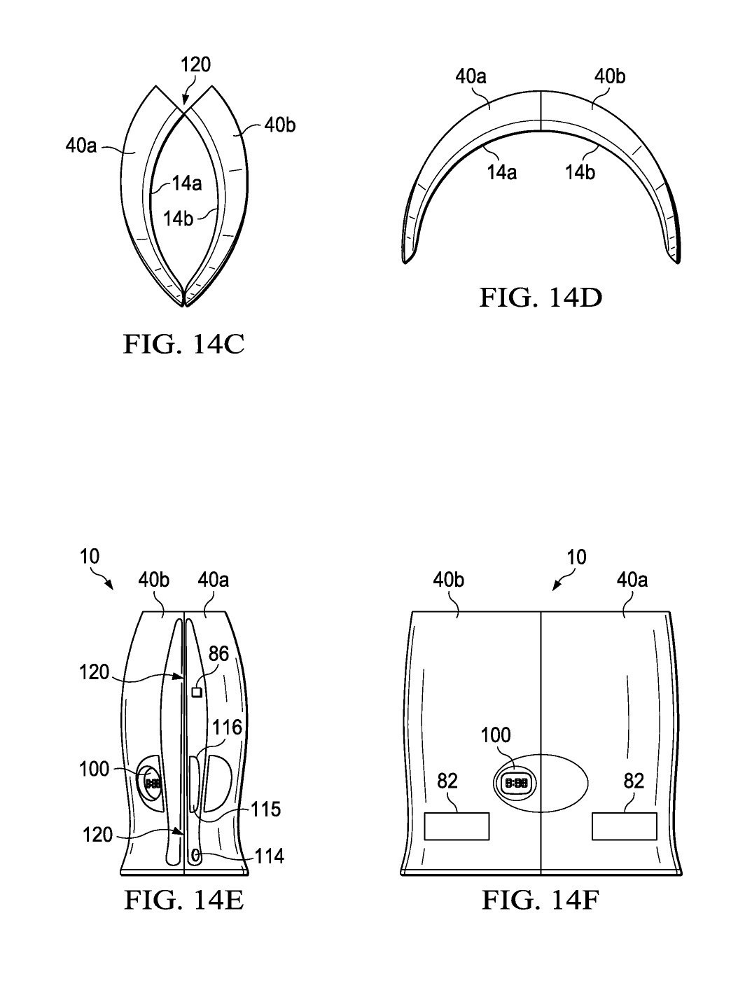

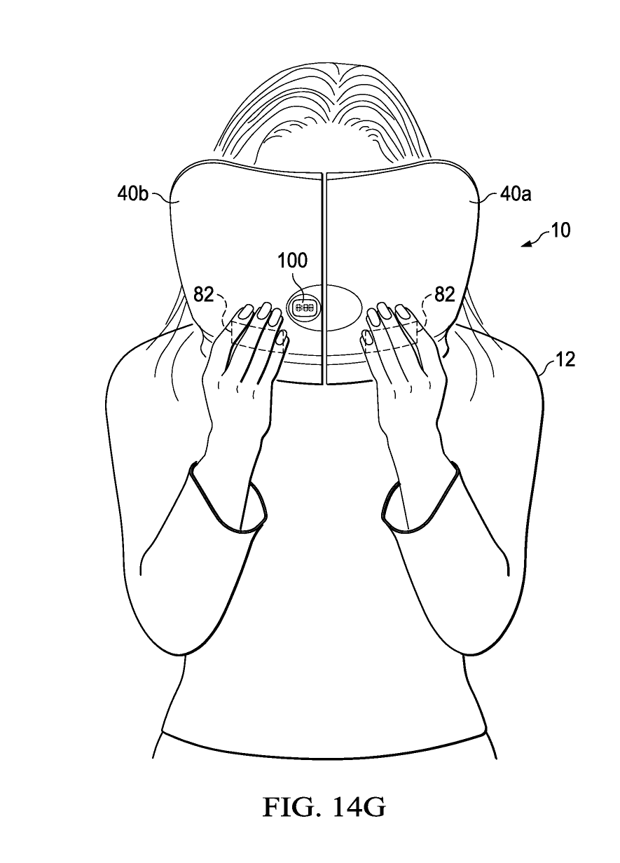

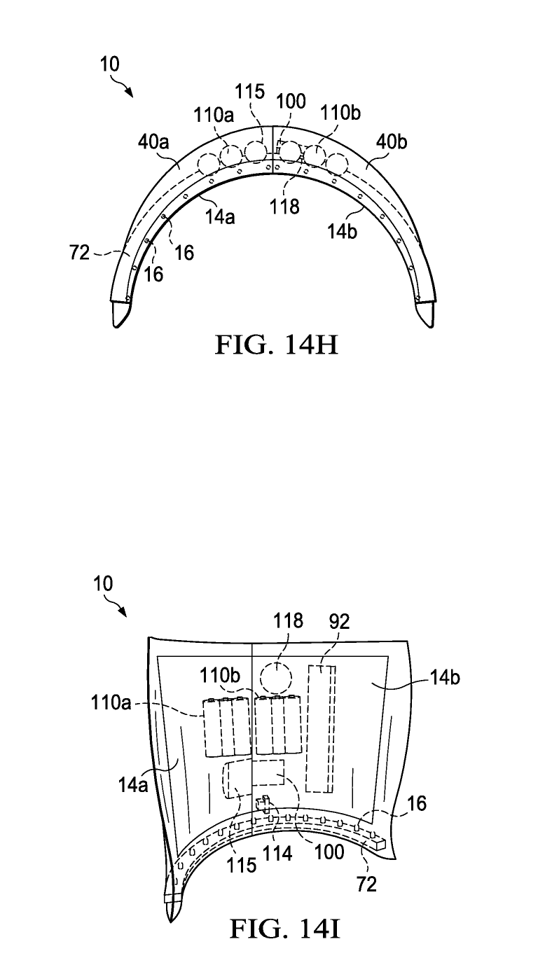

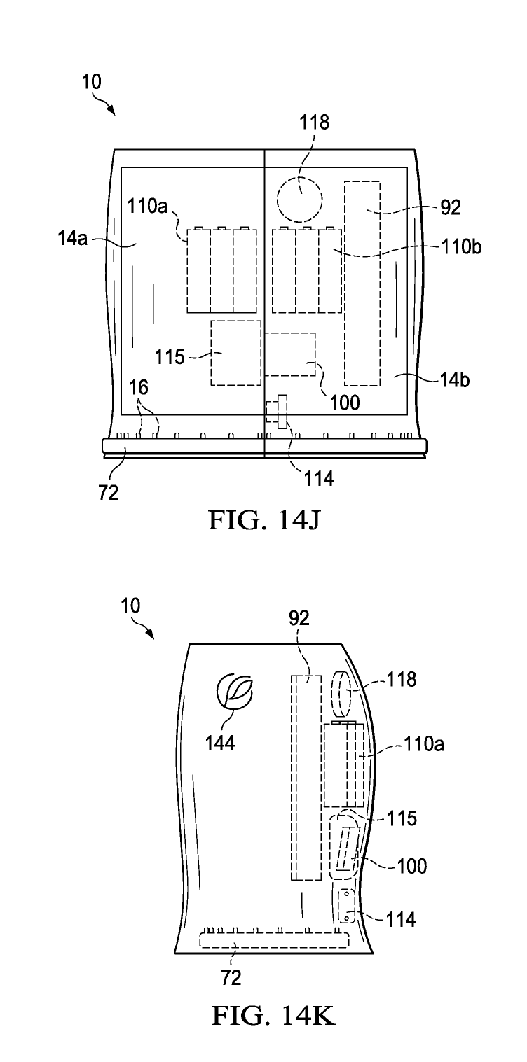

As discussed above, panel 14 may be planar or may include one or more curves, bends, or other non-planar contours. Further, device 10 may include a single panel 14 or multiple panel sections that cooperate in any suitable manner. For example, in the embodiments shown in FIGS. 14, 15, and 18 (discussed below in greater detail), device 10 includes two panel sections 14a and 14b coupled by a hinged connection such that device 10 can be folded between a storage position and a treatment position. Details of panel 14 disclosed herein generally apply also to individual panel sections (e.g., panel sections 14a and 14b). Thus, “panel 14” as used herein refers to the panel of a single-panel device 10, as well as to individual panel sections (e.g., panel sections 14a and 14b) of a multi-panel device 10.

In some embodiments, panel 14 may be substantially planar. In other embodiments, panel 14 may be curved or may include one or more curved portions. More specifically, panel 14 may include one or more concave portions, one or more convex portions, one or more planar portions, or any combination thereof. In some embodiments, panel 14 may be curved to correspond to the curved shape of one or more particular body parts (e.g., the face, hand, thigh, etc.). For example, panel 14 may be generally concave to correspond to the general curve of a user's face, e.g., as shown in the embodiments of FIGS. 7, 8, 9, 14-19, etc. Panel 14 may be concave (or convex) about a single axis (e.g., a partial cylindrical shape) or about multiple different axes (e.g., a partial spherical shape or a bowl shape). In embodiments in which panel 14 is concave (or convex) about multiple different axes, the degree of concavity (or convexity) about each axis may the substantially the same or substantially different.

FIG. 7 is a three-dimensional view of one half of a dual-section device 10 including housing sections 40a and 40b for housing panel sections 14a and 14b, respectively. Specifically, FIG. 7 illustrates housing section 40b with panel section 14b partially covered by a bezel 44. As shown, panel section 14b is concave about a vertical axis A_{V} to for a generally partial-cylinder shape. In this example, a row of LEDs 16 (e.g., yellow-light LEDs alternating with infrared LEDs) is arranged on a heat sink 72 positioned at the bottom edge 24 of panel 14b.

FIGS. 8 and 9 illustrate top views of example devices 10 having example concave panel configurations. In FIG. 8, parabolic panel sections 14a and 14b are housed in housing sections 40a and 40b, which are coupled by a connection 120, to form a parabolic panel 14. Arrows are used to indicate the direction of the emitted light, which may be normal to the concave panel surface.

In FIG. 9, circular panel sections 14a and 14b are housed in housing sections 40a and 40b, which are coupled by a connection 120, to form a semi-circular panel. Again, arrows are used to indicate the direction of the emitted light, which may be normal to the concave panel surface. The semi-circular panel 14 may extend through any angular fraction of a circle, e.g., within the range from 90 to 180 degrees. In the illustrated example, the panel extends through an angle of almost 180 degrees.

Connection 120 may be any type of connection for coupling housing sections 40a and 40b to each other. For example, connection 120 may be a rigid connection, a flexible connection, a pivoting connection (e.g., a hinged connection), a sliding connection, a releasable connection (such that panel sections 14a and 14b may be separated from each other for storage), or any other type of connection.

FIGS. 8 and 9 are examples only, and any other suitable geometric shapes may be used, which may include multiple different curves or contours. Other example curved panel configurations are shown in the embodiments of FIGS. 14-19, 21, 24, 25, and 26. For example, panels 14 shown in FIGS. 14, 15, 16, and 17 are generally concave about a vertical axis A_{V}.

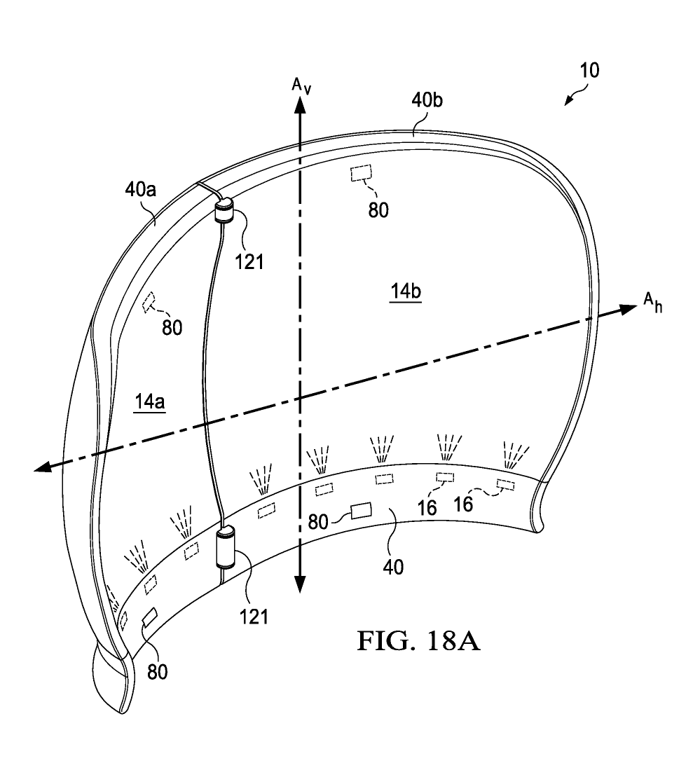

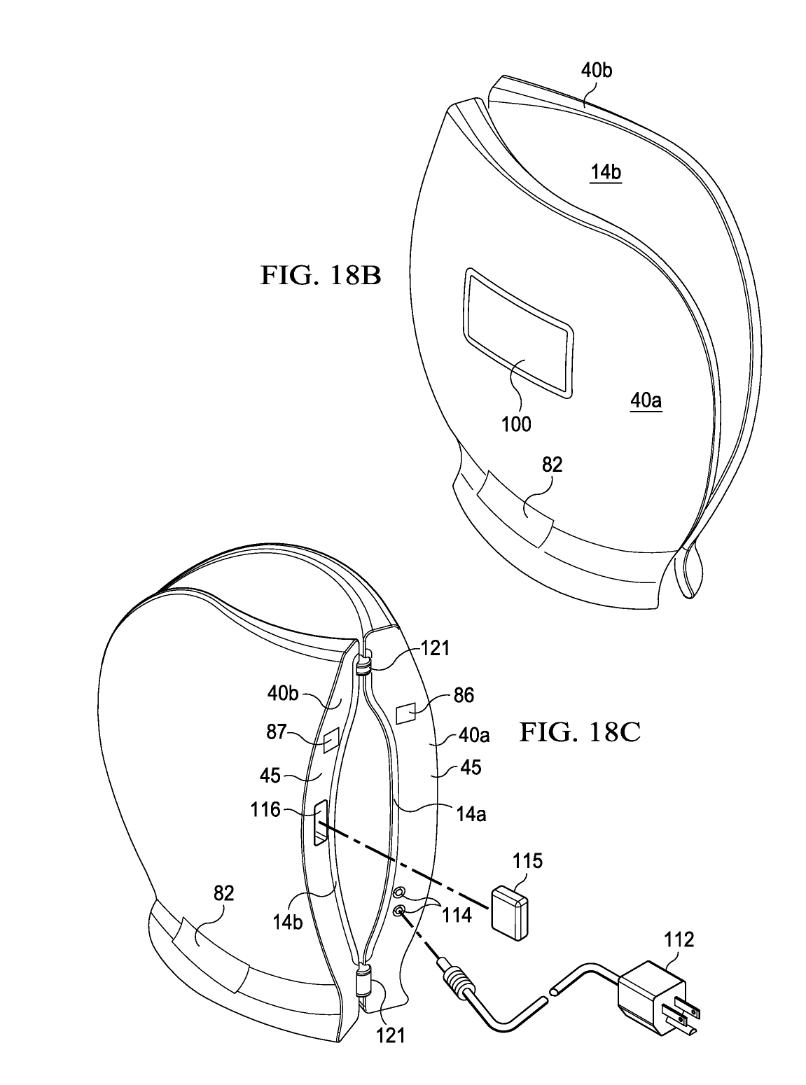

Panel 14 shown in FIG. 18 is generally concave about a vertical axis A_{V} and also concave about a horizontal axis A_{H}, thus forming a generally bowl-like shape. In this embodiment, the degree of concavity about the vertical axis A_{V} is greater than the degree of concavity about the horizontal axis A_{H}, which generally corresponds to the shape of a user's head (which generally curves more from left-to-right than from top-to-bottom). In other embodiments, panel 14 may be equally concave about a vertical axis A_{V} and a horizontal axis A_{H}. For example, the panel 14 shown in FIG. 18 (which generally mimics a vanity mirror) has a regular partial-spherical or partial-parabolic concave shape that is 360 degrees symmetrical. It should be understood that in other embodiments, panel 14 may have any other suitable shape or contours.

As discussed herein, panel 14 may have any an illuminated area 46 of any suitable shape and size. In some embodiments, e.g., for treating the face, the illuminated area 46 of panel 14 may be between 250 and 500 cm². For example, in specific embodiments, e.g., for treating the face, panel 14 may be generally concave about at least one axis and may have an illuminated area 46 between about 300 cm² and about 400 cm². In some embodiments, e.g., for treating the face, panel 14 may be generally concave about at least one axis and may have an illuminated area 46 of about 350 cm². In some embodiments, the illuminated area 46 of a generally concave panel 14 may have a height of about 13-17 cm, an internal side-to-side width (measured along a straight line) of about 15-19 cm, and a internal center arc depth of about 6-10 cm, such that the illuminating area 46 is about 350 cm².

In embodiments in which panel 14 includes two or more panel sections (e.g., 14a, 14b, etc.), each panel section may have the same shape and/or size, or different shapes and/or sizes. For example, in some embodiments, panel 14 includes two panel sections 14a and 14b having the same shape and size, e.g., the example embodiments shown in FIGS. 14, 15, and 18.

In other embodiments, e.g., for spot treating the face or for treating other smaller areas, the illuminated area 46 of panel 14 may be between about 19 and about 78 cm². For example, in some embodiment embodiments, e.g., for spot treating the face or for treating other smaller areas, panel 14 may be generally flat or generally concave about at least one axis and may have an illuminated area 46 of about 1 cm².

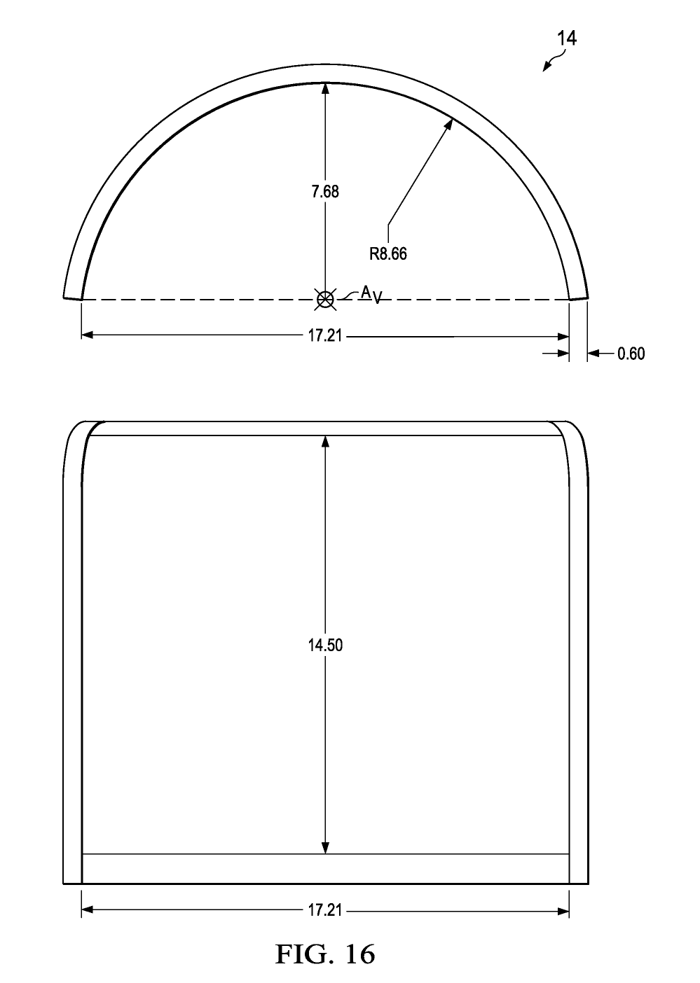

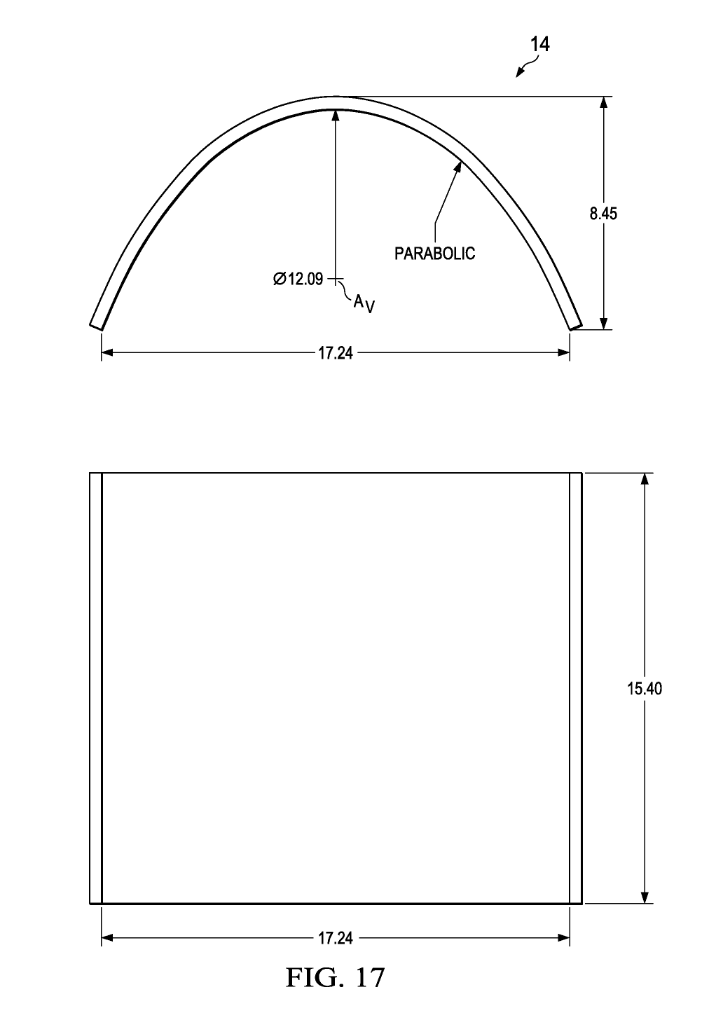

FIGS. 16 and 17 illustrate top and front views of two example panels 14, with example dimensions (in centimeters), according to example embodiments. The panels 14 and dimensions shown in FIGS. 16 and 17 represent the illuminated area 46 of panels 14. Thus, the dimensions shown in FIGS. 16 and 17 represent the portion of the respective panel 14 that is not covered by a housing bezel 44 or other portion of housing 40.

FIG. 16 illustrates top and front views of an example semi-circular panel 14. As shown in the top view, panel 14 is concave about a vertical axis Aᵥ (perpendicular to the page). This example panel 14 has a radius of 8.66 cm, an internal depth of 7.68 cm, an internal side-to-side width of 17.21 cm, and a height of 14.50 cm.

FIG. 17 illustrates top and front views of an example parabolic panel 14. As shown in the top view, panel 14 is concave about a vertical axis Aᵥ (perpendicular to the page). This example panel 14 has an external depth 8.45 cm, an internal side-to-side width of 17.24 cm, and a height of 15.40 cm. It should be understood that the shapes and dimensions shown in FIGS. 16 and 17 are examples only, and that any other suitable shapes and dimensions may be used.

### Sensors

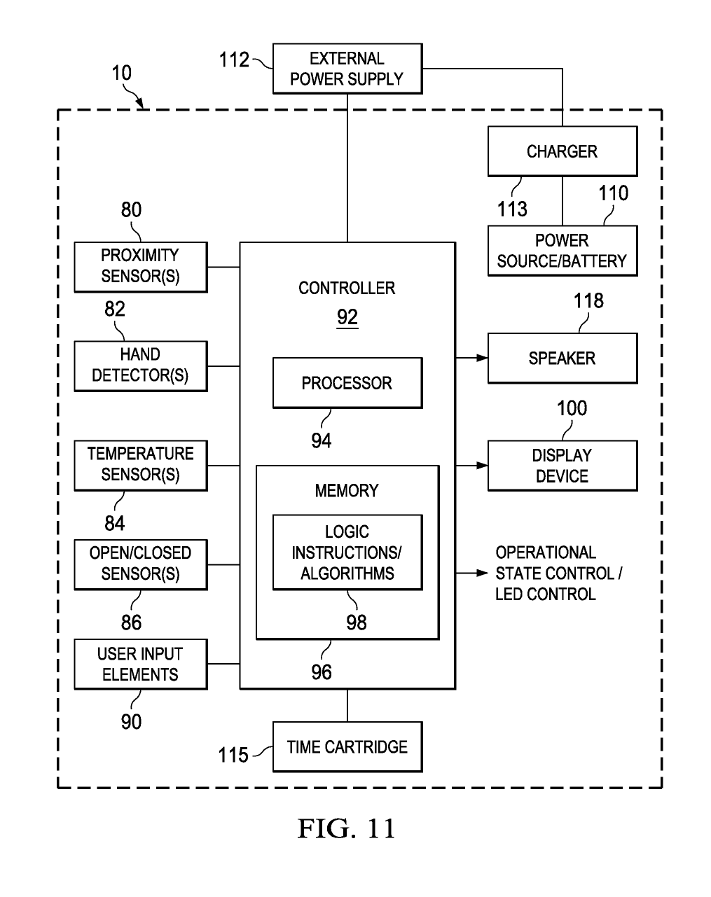

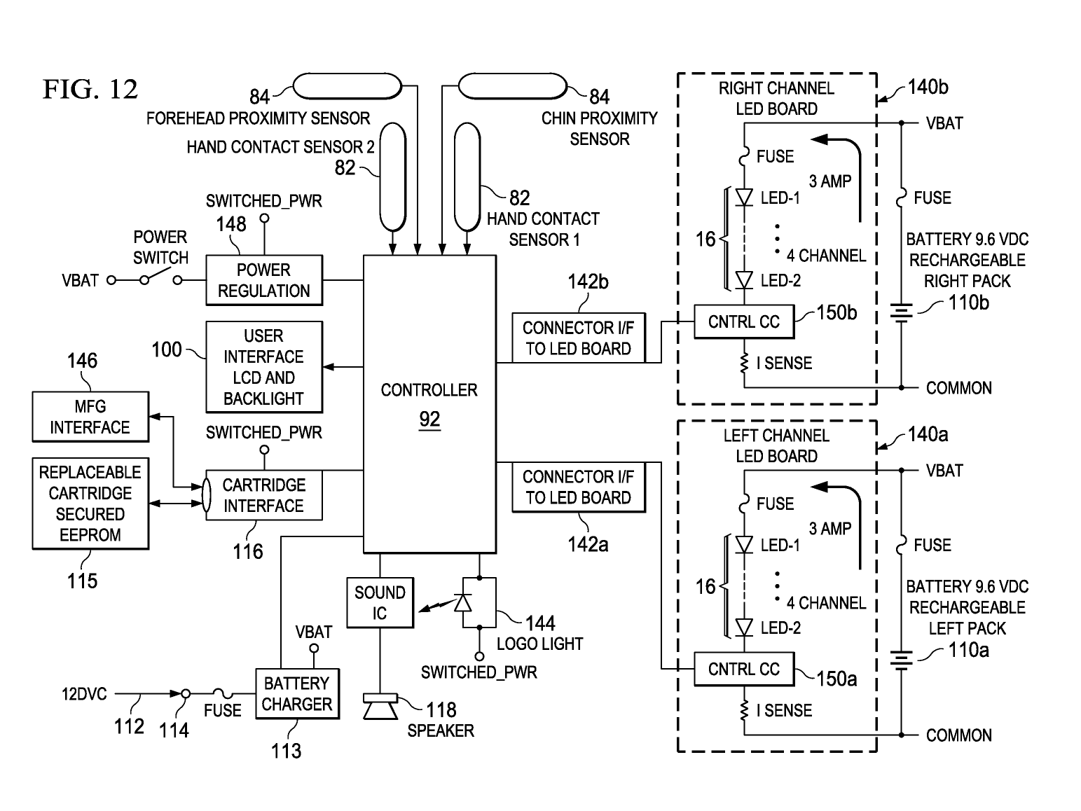

Device 10 may include any number and types of sensors for detecting or measuring one or more various parameters, such as, for example: (a) detecting whether the user 12 is holding device 10, (b) detecting whether device 10 is in a treatment configuration (e.g., for devices with a hinged housing, detecting whether the device is folded open), (c) detecting whether the treatment area of the user 12 is positioned in a predetermined “treatment position” relative to device 10, (d) detecting for an overheated condition, and/or any other suitable types of sensors. As discussed in greater detail below with reference to FIGS. 11 and 12, such sensors may communicate signals to a controller 92 for affecting various operational aspects of device 10, e.g., triggering transitions between different operational states (e.g., an “off” state, a “ready” state, and a “treatment” state), or providing various feedback to the user 12 (e.g., via a display 100).

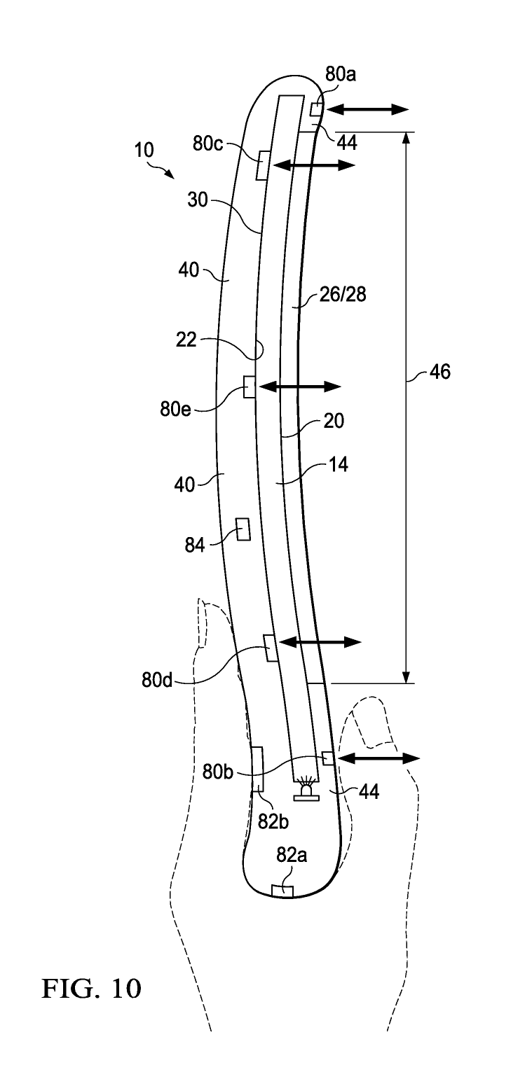

FIG. 10 illustrates a vertical cross-section of an example device 10 including various types and example locations of sensors, in accordance with one embodiment. In this example, device 10 is intended for applying treatment to a user's face, and includes one or more facial proximity sensors 80, one or more hand detectors 82, and one or more temperature sensors 84. In addition, if device 10 is foldable (i.e., hinged), device 10 may include one or more device configuration sensors 86, e.g., as shown in FIG. 18C.

Facial proximity sensors 80 may include any suitable sensor or sensors for detecting or measuring the proximity of the user 12 to device 12. Proximity sensors 80 may be located in any suitable location on device 10. For example, as shown in FIG. 10, one or more proximity sensors may be located near the center of panel 14, e.g., sensor 80e, and/or near an edge of panel 14, e.g., sensors 80a-80d. One or more sensors 80 may be located outside of the illuminating area 46 of panel 14, such as sensors 80a and 80b located in the bezel region 44 of housing 40. Such sensors operate without having to send/receive proximity detection signals through panel 14. Alternatively or in addition, one or more sensors 80 may be located within the illuminating area 46 of panel 14, such as sensors 80c, 80d, and 80e located behind panel 14. Such sensors send/receive proximity detection signals through panel 14. Rear reflector 30 located behind panel 14 may include a hole or opening through which such sensors 80 may send/receive proximity detection signals.

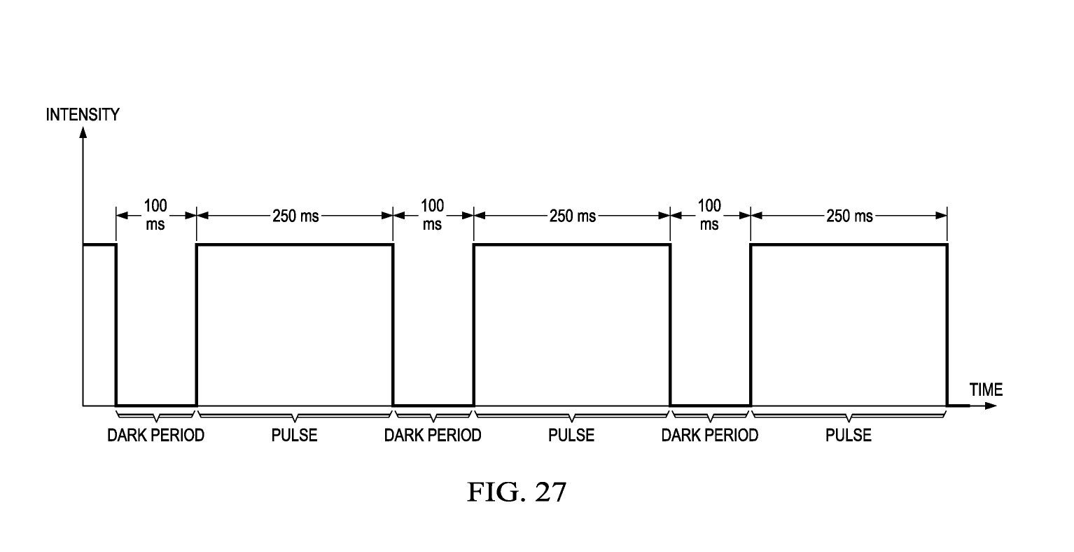

Proximity sensors 80 may take proximity readings at any suitable times, in any suitable frequency and/or pattern, continuously, non-continuously, upon some predetermined triggering event(s), or according to any other algorithm. For example, proximity sensors 80 may begin taking readings once device 10 is turned on, or enters a particular operational state (e.g., “ready” state). Proximity sensors 80 may then continue to take readings at a suitable frequency (e.g., at one second intervals) as long as device 10 remains turned on, or remains in one or more particular operational states (e.g., “ready” state or “treatment” state). In some embodiments in which LEDs 16 are operated in a pulsed manner, proximity sensors 80 may take readings only between LED pulses (e.g., during “dark” periods shown in FIG. 27). Thus, potential interference between the operation of LEDs 16 and proximity sensors 80 may be avoided or reduced. In other embodiments, proximity sensors 80 may take readings LED pulses (e.g., during “pulse” periods shown in FIG. 27). In embodiments with multiple proximity sensors 80, the sensors may take readings simultaneously, or in an alternating manner, or in any other pattern or according to any other algorithm.

In some embodiments, device 10 includes a single proximity sensor 80, for determining a single distance between the user's face and device 12. For example, with reference to FIG. 10, device 10 may include a single proximity sensor 80e positioned near the center of panel 14, or a single proximity sensor 80a positioned near an edge of panel 14. Signals from a single proximity sensor 80 can be processed by controller 92 to determine whether the user's face is within a predetermined acceptable treatment distance from device 10 (e.g., in order for treatment to begin or continue).

In other embodiments, device 10 includes multiple proximity sensors positioned at different locations on device 10, or directed at different angles, or both, in order to determine multiple distances between the user's face and device 10. Signals from multiple proximity sensors 80 can thus be processed by controller 92 to determine the distance between device 10 and multiple different locations on the user's face. Thus, device 10 may determine the distance between the user's face and device 10, as well as the angular orientation and/or lateral positioning of the user's face relative to device 10. For example, a pair of proximity sensors 80a and 80d (or 80c and 80d) located near the top and bottom of device 10, respectively, can be used to determine the up-down angular orientation of the user's face relative to device 10. As another example, a pair of sensors 80 located near the right and left edges of device 10, respectively, can be used to determine the left-right angular orientation of the user's face relative to device 10. Proximity sensors 80a and 80c positioned near the upper portion of device 10 may be referred to as forehead sensors, while proximity sensors 80b and 80d positioned near the lower portion of device 10 may be referred to as chin sensors. Further, three, four, or more proximity sensors 80 positioned at different locations on device 10, or directed at different angles, or both, may be used to determine multiple different angular orientations of the user's face relative to device 10.

Signals from multiple proximity sensors 80 can be processed to determine whether the user's face is within a predetermined acceptable treatment distance from device 10, as well as within predetermined acceptable ranges for one or more angular orientations and/or lateral positioning of the user's face relative to device 10 (e.g., in order for treatment to begin or continue). In practice, controller 92 may compare proximity readings from multiple proximity sensors 80 to predetermined acceptable ranges for each respective sensor, and determine whether the user's face is positioned in a “treatment position” based on the number of proximity readings falling within the corresponding acceptable ranges. For example, controller 92 may require that the readings from all proximity sensors 80 fall within their acceptable ranges, or some other predetermined minimum number (e.g., acceptable readings from 3 out of 4 sensors). Alternatively, controller 92 may calculate values for one or more facial angles or other position indicators based on proximity readings from multiple proximity sensors 80, and then compare such values to predetermined acceptable ranges for each respective value, and determine whether the user's face is positioned in a “treatment position” based on the number of proximity readings falling within the corresponding acceptable ranges (e.g., requiring all or some other predetermined minimum number of acceptable readings, as discussed above).

As discussed above, and shown in FIG. 10, device 10 may include one or more hand detectors 82 to detect whether the user 12 is physically holding device 10, which in some embodiments may be a requirement for beginning a treatment. Each hand detector 82 may include any type of detector known in the art for detecting direct physical contact (or alternatively, for detecting direct or near physical contact) by a person. Device 10 may include any suitable number of hand detectors 82, which may be located at any one or more suitable locations. For example, device 10 may include a single hand detector for detecting either hand, or one hand detector for detecting each hand, or multiple hand detectors for detecting each hand. The example shown in FIG. 10 illustrates two example positions of hand detector positions, indicated at 82a and 82b. The example embodiments shown in FIGS. 14G and 18C each include two hand detector 82, one for each hand.

With reference to FIG. 11, controller 92 may use signals from hand detectors 82 as input for triggering transitions between different operational states (e.g., from an “off” state to a “ready” state and vice versa), or for providing various feedback to the user 12 (e.g., via a display device 100). For example, upon a required hand detection, controller 92 may transition from an “off” state to a “ready” state. As another example, upon a break or termination in the required hand detection (e.g., if the user puts down the device), controller 92 may transition from a “ready” state to an “off” state, or from a “treatment” state to a “ready” state or an “off” state, depending on the relevant operational algorithm. In order to trigger a response (e.g., a transition between states or a display of information), controller 92 may require positive detection readings from only a single hand detector 82, from all hand detectors 82, or from some other predetermined minimum number of sensors 82 (e.g., 3 out of 4).

Hand detectors 82 may take detection readings at any suitable times, in any suitable frequency and/or pattern, continuously, non-continuously, upon some predetermined triggering event(s), or according to any other algorithm. For example, hand detectors 82 may begin taking readings once device 10 is turned on. In some embodiments, hand detectors 82 are continuously detecting sensors.

As discussed above, and shown in FIG. 10, device 10 may include one or more temperature sensors 84 for detecting the temperature at one or more locations in device 10. Temperature sensors 84 may include any suitable types of sensors for measuring temperature. With reference to FIG. 11, controller 92 may monitor signals from one or more temperature sensors 84 to ensure that device 10 is not overheating. For example, controller 92 may compare signals temperature sensor signals to a threshold temperature, and if such thresholds is exceeded, triggering an overheat alarm (e.g., by displaying an alarm to the user via display device 100 and/or generating an audible alarm) and/or automatically turning device 10 off and/or any other suitable response.

In addition, if device 10 is moveable between treatment and non-treatment configurations (e.g., between open and closed configurations), device 10 may include one or more device configuration sensors 86, e.g., as shown in FIG. 18C. Device configuration sensors 86 may include any suitable type of sensor(s) for detecting the current configuration of device 10. For example, device configuration sensor(s) 86 may be configured to detect whether device 10 is in a treatment configuration (e.g., an open configuration) or a non-treatment configuration (e.g., a closed, folded, or disconnected configuration (e.g., for storage of device 10)).

For example, with reference to FIG. 18C, which shows an example device 10 including panel sections 14a and 14b housed in housing sections 40a and 40b, respectively, and shown in a non-treatment (closed) configuration, device 10 may include a configuration sensor 86 on an inner edge 45 of housing section 40a or 40b, for detecting contact or immediate proximity with the opposing inner edge 45 of the other housing section 40a or 40b. In some embodiments, e.g., as shown in FIG. 18C, a metallic or other conductive or reflective element 87 may be located opposite device configuration sensor 86, to facilitate the detection by sensor 86. With reference to FIG. 11, controller 92 may monitor signals from device configuration sensor(s) 86 to determine whether device 10 is in a treatment configuration or a non-treatment configuration. In some embodiments, controller 92 may only allow transition to certain operational states (e.g., a “ready” state and/or a “treatment” state) if controller 92 determines that device 10 is in a treatment configuration. Further, controller 92 may transition between certain operational states (e.g., from a “treatment” state” to a “ready” state, or from “ready” state” to an “off” state, and/or a “treatment” state) if controller 92 determines that device 10 has been moved to a non-treatment configuration.

### Power and Control System Components

Device 10 may include any suitable power and control system for powering and controlling the various operations of the device. FIG. 11 is a block diagram illustrating components of an example power and control system for a device 10 according to certain embodiments. The control system may include, for example, one, some, or all types of sensors 80, 82, 84, and 86, user input elements 90, a controller 92, a display device 100, a time cartridge 115, and a speaker 118. The different types of sensors 80, 82, 84, and 86 are discussed above in more detail.

User input elements 90 may include any elements or devices allowing a user to provide input to device 10, e.g., one or more physical or virtual buttons, switches, knobs, sliders, or other interactive elements. Virtual elements may be provided via one or more interactive touchscreens, which may be integrated with, or separate from, display device 100. User input elements 90 may be provided, for example, for turning the device on or off, resetting the device, adjusting the intensity of LED light treatment, adjusting the duration of a treatment session, locking the device (e.g., preventing the device from automatically turning on in response to hand detection and/or face proximity detection), etc. User input elements 90 may include a wake-up button to signal a transition from a low-power “sleep” state into a “ready” state.

Controller 92 may include any suitable electronic components for controlling the operation of device 10, such as controlling a display device 100, controlling the current operational state of device 10 (e.g., transitioning between “off”, “ready”, and “treatment” states), and generating alarms. For example, controller 92 may include a processor 94 and memory 96. Memory 96 may store algorithms or other program instructions 98, e.g., as embodied in software and/or firmware, that are executable by processor 94 for providing any of the functionality of device 10 discussed herein. Processor 94 may comprise any system, device, or apparatus operable to interpret and/or execute software or program instructions and/or process data associated with device 10 (e.g., user input received via user input elements 90 and/or signals from various sensors 80, 82, 84, and 86), and may include, without limitation, a microprocessor, microcontroller, digital signal processor (DSP), application specific integrated circuit (ASIC), or any other digital or analog circuitry. Controller 92 be programmed to transition from a low-power “sleep” state to a “ready” state based on an input from user input element 90 and to transition from the “ready” state to a low-power “sleep” state after a period of inactivity.

Memory 96 is communicatively coupled to processor 94 and may include any computer-readable media suitable for storing any data or logic associated with device 10, including program instructions 98. For the purposes of this disclosure, computer-readable media may include any instrumentality or aggregation of instrumentalities that may retain data and/or instructions for a period of time, e.g., random access memory (RAM), read-only memory (ROM), electrically erasable programmable read-only memory (EEPROM), and/or flash memory; and/or any combination of such memory types.

Program instructions 98 may define any suitable rules, algorithms, or logic for controlling the operation of device 10. As one example, program instructions 98 may define rules or algorithms that specify when and how controller 92 transitions between different operational states of device 10, e.g., defining when and how to transition between “off”, “ready”, and “treatment” states based on various inputs, such as user input received via user input elements 90 and/or signals from various sensors 80, 82, 84, and 86, for example. Program instructions 98 may be embodied as software, firmware, or a combination thereof.

Display device 100 may include any suitable device for electronically displaying information to user 12. For example, display device 100 may comprise an LCD display, an active matrix display (e.g., a thin-film transistor (TFT) display), an LED display, etc. Display device 100 may be located at any suitable location on device 10. For example, display device 100 may be integrated into the rear housing of device 10, e.g., as shown in the embodiments of FIGS. 14, 15, and 18. In some embodiments, device 10 may include multiple display devices 100 positioned at different locations.

Time cartridge 115 may comprise a removable cartridge that records (e.g., using a timer or counter) the treatment time or number of treatment sessions provided by device 10 over time. Time cartridge 115 may specify a preset treatment time and/or number of treatment sessions that can be provided by device 10 before the cartridge must be replaced with a new cartridge (or replenished with more treatment time or treatment sessions). Time cartridge 115 may maintain a running total of the treatment time and/or number of treatment sessions remaining on cartridge 115 by decrementing the remaining treatment time or remaining number of sessions during or after each treatment session. For example, time cartridge 115 may specify a preset treatment time of 30 minutes. After each treatment session (e.g., 30 seconds), the remaining treatment time is decremented by 30 seconds and recorded, such that after five treatments the remaining treatment time recorded on cartridge 115 is 27:30. In some embodiments, the treatment time maintained on cartridge 115 is only decremented after a successfully completed session. In other embodiments, the treatment time is decremented by any period of treatment time, even if the treatment is interrupted and not completed. The remaining treatment time or number of sessions for the currently inserted cartridge 115 may be displayed on display device 100, as discussed below.

In other embodiments, rather than decrementing the remaining treatment time or remaining number of sessions on time cartridge 115, time cartridge 115 may maintain an incremented running total of the treatment time and/or number of treatment sessions on cartridge 115 by incrementing the used treatment time or number of used sessions during or after each treatment session. In such embodiments, the time cartridge 115 must be replaced with a new cartridge (or replenished with more treatment time or treatment sessions) once the incremented running total treatment time and/or number of treatment sessions reaches a predetermined number.

In some embodiments, device 10 may be configured such that it can only operate (or at least, enter the treatment state) if a time cartridge 115 having remaining treatments time or sessions is connected to/inserted into device 10 as required. Thus, when the specified treatment time or number of treatment sessions for time cartridge 115 have been used up, device 10 will no longer provide treatments, and the user must replace or replenish the cartridge. In addition, time cartridge 115 may be used as a security or safety device, or essentially as a “key”, wherein the user removes time cartridge 115 from device 10 in order to prevent others from using device 10.

Time cartridge 115 includes a memory device (e.g., non-volatile memory such as flash memory or EEPROM) for storing data including the remaining or used treatment time or number of sessions. In some embodiments, time cartridge 115 also includes a electronics for managing the running total of the treatment time and/or number of sessions, e.g., by decrementing or incrementing the treatment time or number of sessions. Such electronics may include a processor or microprocessor and a timer (if remaining treatment time is recorded). Time cartridge 115 may thus communicate with controller 92 to identify relevant events (e.g., device 10 entering/exiting the treatment state, or successful completion of a treatment), trigger its internal timer (if included), and update the remaining treatment time or number of sessions as appropriate.

In other embodiments, the remaining treatment time or number of sessions is stored on time cartridge 115, but controller 92 performs the actual management of the running total of the treatment time and/or number of sessions, e.g., by decrementing or incrementing the treatment time or number of sessions. Thus, controller 92 may read a value stored on time cartridge 115, determine the updated (e.g., decremented or incremented) value, and write the updated value over the old value on time cartridge 115.

Display device 100 may be configured to display any suitable types of information, such as, for example, current operational state, treatment time, battery status, cartridge status, alarms, etc. An example display device 100 is shown in FIG. 20. In this example, display device 100 displays a battery status indicator 130, a treatment time indicator 132 (indicating either the elapsed or remaining time in the current treatment), and a cartridge status indicator 134 (indicating the remaining treatment time (in minutes) on time cartridge 115). In other embodiments, display device 100 may display any other suitable types of information.

In example embodiments, display device 100 may be configured to display or indicate some or all of the following data:

(1) the condition of battery/batteries 110, including (a) the charge level and/or (b) whether the battery needs charging before usage of device 10;

(2) the condition of cartridge 115, including (a) the number of minutes remaining, and/or (b) whether the cartridge is empty (i.e., out of minutes) and needs replacing/replenishing;

(3) details regarding a current treatment, including (a) displaying a running timer as a treatment progresses, and/or (2) maintaining a display of the last treatment time for a period of x minutes after completion; and/or

(4) when the cartridge 115 is empty (i.e., out of minutes), or if there is no cartridge 115 in device 110, an image of the cartridge flashes and a “Replace Cartridge” message or indication is displayed.

One or more speakers 118 may be provided to provide audio feedback to user 12 for various events, such as the transition between different operational states, the beginning of a treatment, the completion of a treatment, an overheat condition, etc. Speaker(s) 118 may be positioned at any suitable location on device 10, e.g., on the front or back of housing 40 at location(s) designed to be near the user's ear(s) when in use.

The power system for device 10 may include any one or more suitable power sources. For example, device 10 may include one or more batteries 110 and/or an external power source 112. Batteries 110 may include any suitable number and types of disposable batteries (e.g., alkaline batteries) and/or rechargeable batteries (e.g., nickel-cadmium (NiCd), nickel-zinc (NiZn), nickel metal hydride (NiHM), or lithium-ion (Li-ion) batteries), which may have any suitable size, capacity (mAh), and nominal voltage (V). For example, in one embodiment, device 10 includes two 9.6 V DC rechargeable battery packs.

External power source 112 may be used to provide power to operate device 10 and/or to recharge batteries 110 via a charger 113. In one embodiment, device 10 includes six AA batteries in two groups of three-battery series, and a two or three-pronged wall-outlet charger including an AC/DC converter, for recharging the batteries. In some embodiments, device 10 is configured to prevent operation (or at least treatment) when batteries 110 are charging.

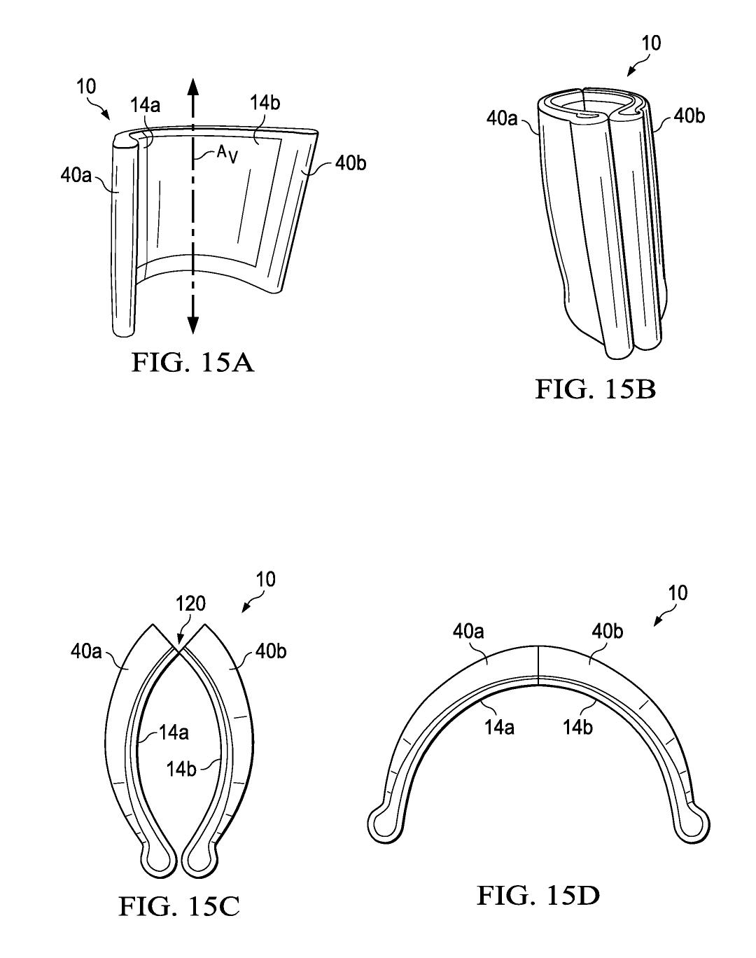

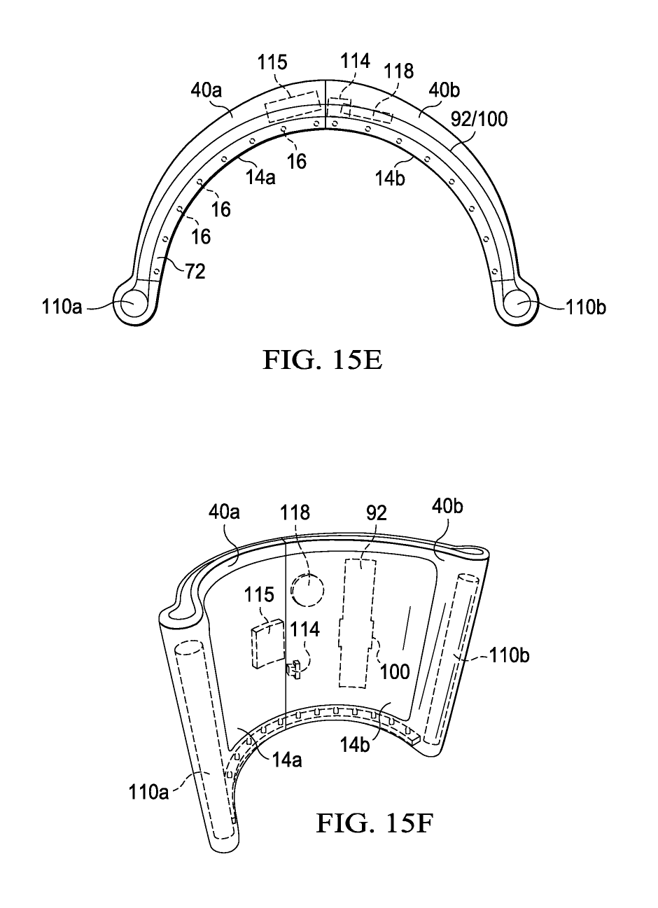

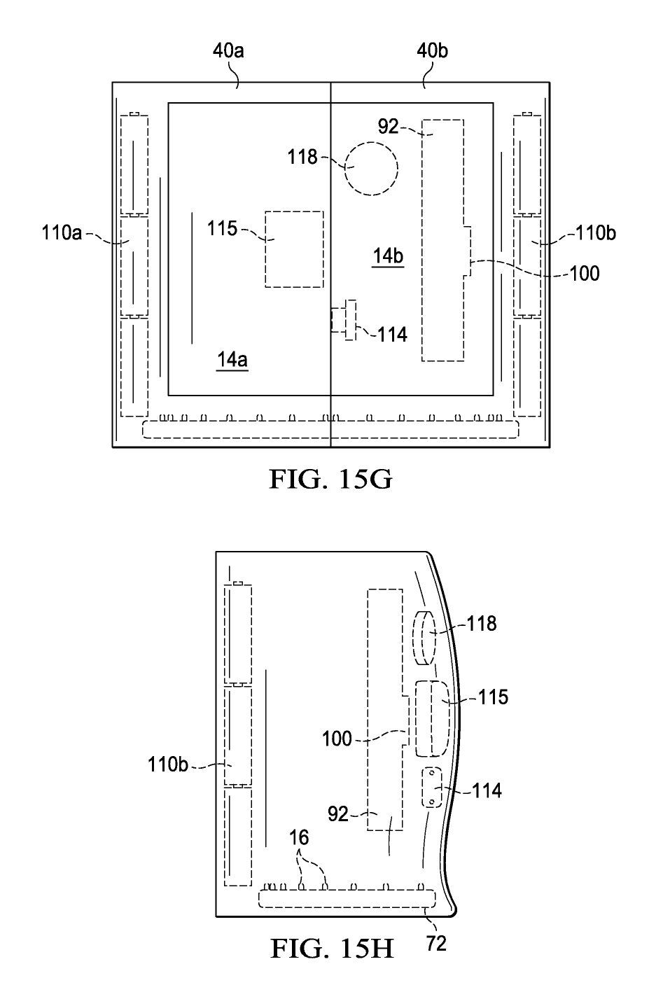

Batteries 110 may be located in any suitable location within device 10. In some embodiments, one or more batteries 110 may be positioned in the housing 40 along or outside one or more edges 24 of panel 14. For example, one or more batteries 110 may be positioned beyond the left and right edges 24 of panel 14, e.g., batteries 110a and 110b shown in FIGS. 15E, 15F, and 15G. As another example, one or more batteries 110 may be positioned in the lower portion of the housing 40 below panel 14. As another example, one or more batteries 110 may be positioned in the upper portion of the housing 40 above panel 14.

In some other embodiments, one or more batteries 110 may be positioned within housing 40 generally behind panel 14. In embodiments with multiple panel sections, one or more batteries 110 may be positioned generally behind one, some, or all panel sections. For example, FIGS. 15E, 15F, and 15G illustrate an embodiment with two panel sections 14a and 14b, wherein batteries 110a and 110b may be positioned in housing sections 40a and 40b behind panel sections 14a and 14b.

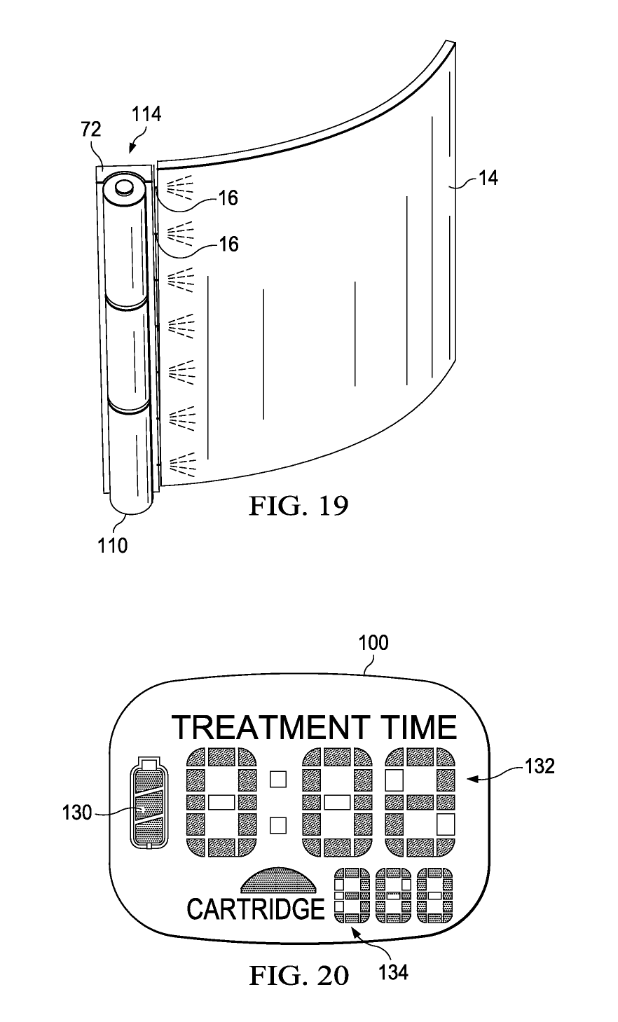

As another example, one or more batteries 110 may be positioned along a central region between left and right panel sections, e.g., as shown in FIG. 19. In the example embodiment of FIG. 19, a vertical center region 114 of device 10 is provided between two panel sections 14a and 14b (panel section 14b is not shown). The vertical center region 114 may house a row of batteries 110 arranged in series, a row of LEDs 16 on each side for illuminating each of the two panel sections 14, and a heat sink 72 for dissipating or transferring heat from LEDs 16 and batteries 110.

### Example System Schematic

FIG. 12 is a block diagram illustrating the operational components of an example device 10, according to an example embodiment. As shown, device 10 includes various electronics connected to controller 92, including:
chin and forehead proximity sensors 84,
a pair of hand contact sensors 82,
right and left channel LED boards 140a and 140b, connected via connector interfaces 142a and 142b, respectively,
a lighted logo 144,
a speaker 118,
a battery charger 113,
a cartridge interface 116 for receiving a time cartridge 115 or connection to a manufacturer interface 146,
an LCD user interface 100, and
a power regulation circuit 148.

Right and left channel LED boards 140a and 140b correspond to right and left panel sections 14 of device 10. Specifically, right channel LED board 140a includes a circuit having a number of right-panel LEDs (e.g., 4 yellow light LEDs and 4 infrared LEDs) powered by a battery pack 110a, a LED controller 150a, and various other electronics (e.g., fuses, resistors, capacitors, etc.) for controlling the operation of the right-panel LEDs. Similarly, left channel LED board 140b includes a circuit having a number of left-panel LEDs (e.g., 4 yellow light LEDs and 4 infrared LEDs) powered by a battery pack 110b, a LED controller 150b, and various other electronics (e.g., fuses, resistors, capacitors, etc.) for controlling the operation of the left-panel LEDs. LED controllers 150a and 150b control the operation of the right-panel LEDs and left-panel LEDs, respectively, based on signals received from controller 92, e.g., by providing a pulsed 3A current as specified by controller 92.

Battery packs 110a and 110b may include any suitable type(s) of batteries for providing power to device 10. In this example, each battery pack 110a and 110b is a rechargeable 9.6V DC battery pack.

Lighted logo 144 may be a logo, symbol, or trademark of a company associated with the device (e.g., manufacturer, seller, retailer, etc.) lighted by LED or other suitable light. Device 10 may be configured to provide operational feedback via lighted logo 144, e.g., by controlling the color, intensity, or other aspect of the lighted logo to indicate the current operational state. For example, in one embodiment, lighted logo 144 may be configured to light steady when device 10 is powered on (and not being charged), pulse when device 10 is being charging, and then stop pulsing (i.e., light steady) when the battery is fully charged.

Cartridge interface 116 may comprise any interface for receiving or connecting to a replaceable time cartridge 115. As shown, cartridge interface 116 may also be configured for interfacing with a manufacturer interface 146 allowing a manufacturer to communicate with controller 92 (e.g., for testing or updating software or firmware).

Battery charger 113 is coupled to a power port 114 for receiving an external power source 112, e.g., a 12V DC power source.

Power regulation circuit 148 may be configured to regulate the power applied to controller 92 from battery pack 110a and/or 110b.

### System Operation

As discussed above, device 10 may be configured to deliver low-level light therapy to user 12 using an LED edge-lit panel 14. Further, as discussed above, controller 92 controls the operation of device 10, such as controlling a display device 100, controlling the current operational state of device 10 (e.g., transitioning between “off”, “ready”, and “treatment” states), and generating alarms. Controller 92 makes control decisions by applying various rules, algorithms, or other logic 98 (as embodied in software and/or firmware) to various input data, such as input data received from user 12 via user input elements 90 and/or signals received from various sensors 80, 82, 84, and 86.

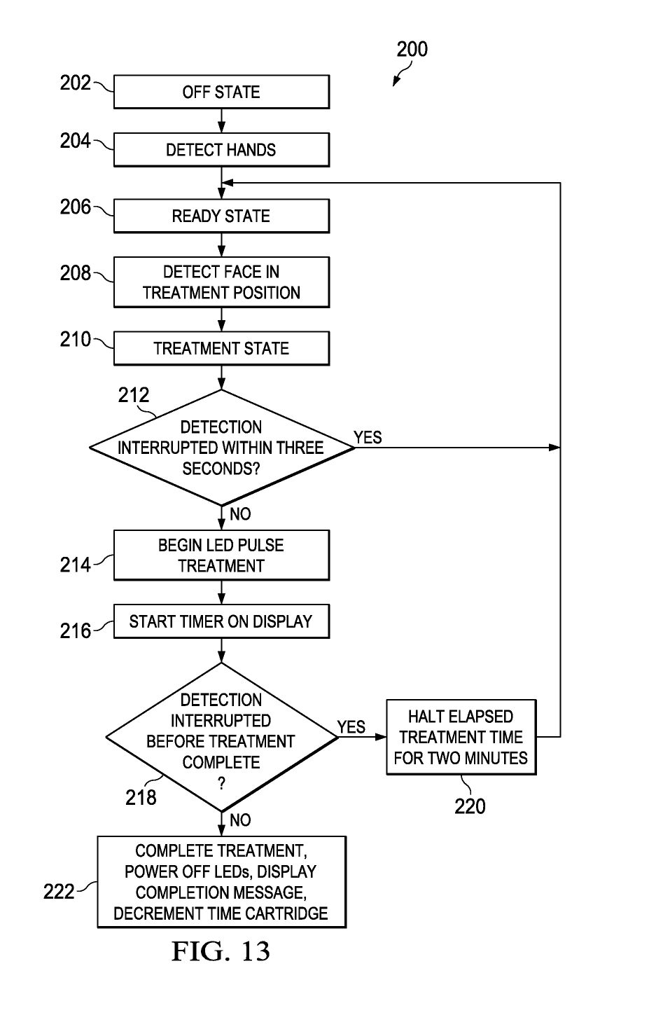

To illustrate, one example set of rules is listed below, and then discussed in the context of an example treatment process flow, illustrated in FIG. 13. The example set of rules define the logic for the controller's selection of/transition between three different operational states: an “off” state, a “ready” state, and a “treatment” state. In the “off state, device 10 is powered off In the “ready” state, device 10 has partially powered up, but the LEDs do not operate as the treatment-level intensity. In the “treatment” state, LEDs operate as the treatment-level intensity.

The example rules provide:

(a) Device 10 enters the “ready” state if hand detectors 82 detect the presence of both hands.

(b) The “ready” state is indicated by a lower power continuous panel glow mode. Thus, LEDs 16 are operated such that panel 14 glows at an intensity substantially lower than the treatment intensity.

(c) The “ready” state is further indicated by an illuminated backlight of display 100.

(d) When proximity sensors 80 detect that the user's face is in a proper treatment position relative to panel 14, device 10 transitions from the “ready” state to the “treatment” state by softly increasing the LED intensity to the treatment intensity over a period of approximately one second.

(e) If either the hand or proximity detection is interrupted within the first three seconds after entering the “treatment” state, device 10 returns to the “ready” state and the LED intensity is reduced accordingly.

(f) Entry into the “ready” state always occurs softly over a period of approximately one second.

(g) If activation is maintained (i.e., no interruption of hand or proximity detection) for more than approximately three seconds, a “welcome” or “treatment has started” sound is played through speaker 118 and a treatment time begins to increment on display 100, and LEDs 16 begin the pulse treatment (at 3 Hz).

(h) A treatment time of 35 seconds is predefined.

(i) If either the hand or proximity detection is interrupted before the treatment is completed, device 10 returns to the “ready” state and the LED intensity is reduced accordingly. The elapsed treatment time shown on display 100 is suspended, and display 100 remains lit with the suspended elapsed treatment time displayed for two minutes.

(j) If activation is maintained (i.e., no interruption of hand or proximity detection) for the full treatment time (35 seconds), LEDs 16 are powered off, a “congratulations” or “treatment has concluded” sound is played through speaker 118, and display 100 remains lit with the elapsed treatment time (35 seconds) displayed for two minutes.

It should be understood that the rules and specific time durations listed above are examples only and particular embodiments may include various other or additional rules and any other suitable time durations.

FIG. 13 illustrates an example treatment process flow 200, according to the example set of rules (a)-(j) listed above, according to one example embodiment. Device begins in the “off” state (i.e., powered off), indicated at 202. When hand detectors 82 detect the presence of both hands holding device 10, indicated at 204, controller 92 transitions device 10 from the “off” state to the “ready” state, indicated at 206. As discussed above, the “ready” state is indicated by a lower power continuous panel glow mode. Thus, LEDs 16 are operated such that panel 14 glows at an intensity substantially lower than the treatment intensity. Also, display 100 is backlit.

When proximity sensors 80 detect that the user's face is in a proper treatment position relative to panel 14, indicated at 208, controller 92 transitions device 10 from the “ready” state to the “treatment” state, indicated at 210. As discussed above, controller 92 transitions device 10 from the “ready” state to the “treatment” state by softly increasing the LED intensity to the treatment intensity over a period of approximately one second.

If either the hand or proximity detection is interrupted (as detected by sensors 80 or 82) within the first three seconds after entering the “treatment” state, controller 92 transitions device 10 from the “treatment” state back to the “ready” state, as indicated at 212. If not (i.e., if activation is maintained for three seconds), a “welcome” or “treatment has started” sound is played through speaker 118 and a treatment time begins to increment on display 100, and LEDs 16 begin the pulse treatment (at 3 Hz), indicated at 214 and 216. A treatment time of 35 seconds is predefined in this example.

If either the hand or proximity detection is interrupted (as detected by sensors 80 or 82) before the treatment is completed, indicated at 218, controller 92 transitions device 10 from the “treatment” state back to the “ready” state and the LED intensity is reduced accordingly. The elapsed treatment time shown on display 100 is suspended, and display 100 remains lit with the suspended elapsed treatment time displayed for two minutes, as indicated at 220. Alternatively, if activation is maintained for the full treatment time (i.e., no interruption of hand or proximity detection for the 35 seconds of treatment), LEDs 16 are powered off, a “congratulations” or “treatment has concluded” sound is played through speaker 118, and display 100 remains lit with the elapsed treatment time (35 seconds) displayed for two minutes. In addition, the remaining treatment time and/or number of sessions stored on time cartridge 115 may be decremented and shown on display 100. In some embodiments, the remaining treatment time on time cartridge 115 may also be decremented an appropriate amount of time for incomplete treatments (e.g., where the treatment is interrupted at step 218).

### Pulse Therapy

As discussed above, device 10 may apply a pulsed LED therapy. Thus, controller 92 may operate LEDs 16 in a pulsed manner for the duration of the treatment session (e.g., 35 seconds). FIG. 27 illustrates an example waveform for a pulsed LED treatment, according to one embodiment. As shown, the waveform alternates between 250 ms “pulse” periods in which LEDs 16 are activated and 100 ms “dark” periods in which LEDs 16 are not activated. This waveform corresponds to a pulse frequency of about 3 Hz.

It should be understood that other pulse frequencies may be used in other embodiments. In particular, the duration of the “dark” periods, the duration of the “pulse” periods, and the duty cycle (i.e., the ratio of “dark” period duration to “pulse” period duration), may be selected as desired for the particular application or treatment methodology.

In embodiments that include multiple different types of LEDs 16 (e.g., one or more yellow light LEDs and one or more infrared LEDs), the different types of LEDs 16 may be operated according to the same waveform applied simultaneously, or according to the same waveform but offset by some time period, or according to different waveforms (e.g., waveforms having different pulse durations, dark durations, and/or duty cycles). Further, in some embodiments, one type of LEDs may be pulsed, while another type of LEDs may be applied continuously. For example, yellow light LEDs may be pulsed while infrared LEDs are applied continuously.

### LED Power Levels and LED Density

As discussed above, LEDs 16 may include one type of LED or multiple different types of LEDs. For example, all LEDs 16 may be yellow light LEDs that primarily emit yellow light at a wavelength of about 590 nm. In additional, in some embodiments, some or all LEDs 16 may primarily emit yellow light at a wavelength of about 590 nm and also simultaneously emit a secondary infrared emission of about 870 nm.

In other embodiments, LEDs 16 may include one or more yellow light LEDs that primarily emit yellow light and one or more infrared LEDs that primarily emit infrared light. For example, in some embodiments, a device 10 may include one or more yellow light LEDs that primarily emit yellow light at a wavelength of 590 nm+/−10 nm and one or more infrared LEDs that primarily emit infrared light at a wavelength of 870 nm+/−20 nm. Such embodiments may include any suitable number of each type of LED to provide the desired intensity of yellow light and infrared light to the user. For example, device 10 may include a suitable number of yellow light LEDs to provide a generally uniform (over the illuminating area 46) yellow light intensity of about 4 mW/cm² and a generally uniform (over the illuminating area 46) infrared light intensity of about 0.56 mW/cm². In one embodiment, nine yellow LEDs may provided on each half panel along bottom edge 24d. In another embodiment, twelve infrared LEDs may be provided on each half panel along bottom edge 24d. Thus, LEDs may provide a total output of 4 mW/cm² at 590 nm and 0.56 mW/cm² at 870 nm, and may for example pulse at a rate of about 3 Hz with a duty cycle of about 70% or 75%.

In another embodiment, each half panel may include 4 yellow LEDs and 15 IR LEDs such that device 10 may provide a generally uniform (over the illuminating area 46) yellow light intensity of about 1.87 mW/cm² and a generally uniform (over the illuminating area 46) infrared light intensity of about 0.37 mW/cm².

In yet another embodiment, each half panel may include 9 yellow LEDs and 25 IR LEDs such that device 10 may provide a generally uniform (over the illuminating area 46) yellow light intensity of about 4.21 mW/cm² and a generally uniform (over the illuminating area 46) infrared light intensity of about 0.61 mW/cm².

In still another embodiment, each half panel may include 5 yellow LEDs and 30 IR LEDs such that device 10 may provide a generally uniform (over the illuminating area 46) yellow light intensity of about 2.34 mW/cm² and a generally uniform (over the illuminating area 46) infrared light intensity of about 0.73 mW/cm².

Each of these embodiments may utilize LEDs with any or all of the following characteristics. For the yellow LEDs, the following characteristics are based on an ambient temperature of about 25° C. and a forward input current of about 400 mA. The yellow LEDs may have a peak wavelength of about 597 nm and dominant wavelength between about 583 nm and about 595 nm, e.g., about 590 nm. The yellow LEDs may also have a spectral bandwidth at 50% of about 18 nm. The yellow LEDs may be selected from two brightness groups. The lowest brightness group may generate a luminous flux of approximately 33000 to 39000 mlm and a luminous intensity of about 12000 mcd. The highest brightness group may generate a luminous flux of approximately 71000 to 82000 mlm and a luminous intensity of about 25500 mcd. Table 1 discloses an example set of characteristics for yellow LEDs in the highest and lowest brightness groups arranged in channels of three LEDs arranged in series, according to example arrangements

For the IR LEDs, the following characteristics are based on an ambient temperature of about 25° C. and a forward input current of about 50 mA. The IR LEDs may each produce a radiant on-axis intensity ranging from about 9 mW/sr to about 30 mW/sr, e.g., about 17 mW/sr. The IR LEDs may produce light with a peak wavelength between about 850 nm and about 900 nm, e.g., about 875 nm. The IR LEDs may produce light with a spectral width at full width, half maximum of about 37 nm. Table 1 also discloses characteristics of IR LEDs arranged in channels of three LEDs arranged in series, according to example arrangements.

In some embodiments, device 10 may have an output efficiency of at least 50%, where output efficiency is defined as the total LED light output from the illuminating area 46 divided by the total output from the LEDs of device 10. In other words, the output efficiency is a measure of the portion of the LED emitted light that is actually projected out of the front surface of panel 14 within illuminating area 46, as opposed to the portion of the LED emitted light that is absorbed, escapes, or is otherwise not projected out of the front surface of panel 14 within illuminating area 46.

One advantage of the LED edge-lit designs disclosed herein, as compared to existing rear-lit designs that include a large array of LEDs behind a transparent or semi-transparent panel, is that the individual LEDs may be operated at a higher power (i.e., by applying a higher current), and thus fewer LEDs are needed to provide the same overall output flux. For example, in some embodiments, over 100 milliamps may be applied to each LED 16. In some embodiments, over 500 milliamps may be applied to each LED 16. In particular embodiments, about 1 amp or even greater than 1 amp may be applied to each LED 16.

TABLE 1

| | Yellow | Yellow | |
|---|---|---|---|
| | (Highest | (Lowest | |
| | brightness | Brightness | |
| | group) | group) | IR |
| Voltage/LED (V) | 2.6 | 2.6 | 1.6 |
| Peak Current/LED (A) | 0.4 | 0.85 | 0.07 |
| Duty cycle | 0.71 | 0.71 | 0.71 |
| PS Voltage (V) | 9 | 9 | 9 |
| Number of LEDs | 9 | 9 | 12 |
| Number of FETs | 3 | 3 | 3 |
| Peak Optical Power/LED (W) | 0.135 | 0.135 | 0.019 |
| Average Current/LED (A) | 0.284 | 0.604 | 0.050 |
| Average Optical Power/LED (W) | 0.096 | 0.096 | 0.013 |
| E/O conversion efficiency | 13.0% | 6.1% | 17.0% |
| Heat from 1 LED (W) | 0.643 | 1.473 | 0.066 |
| Heat from 1 FET (W) | 0.341 | 0.724 | 0.209 |
| FET Voltage (V) | 1.200 | 1.200 | 4.200 |
| FET Current (A) | 0.284 | 0.604 | 0.050 |
| Heat power onto heatsink from | 5.78 | 13.26 | 0.79 |
| LEDs (W) | | | |
| Heat power from FETs (W) | 1.02 | 2.17 | 0.63 |
| Device on time (sec.) | 35 | 35 | 35 |
| Total Peak optical power at engine/ | 1.215 | 1.215 | 0.228 |
| panel (W) | | | |
| Total average optical power at | 0.86 | 0.86 | 0.16 |
| engine/panel (W) | | | |
| Panel transmission | 0.4 | 0.4 | 0.4 |
| Panel area (single panel) (cm²) | 175.0 | 175.0 | 175.0 |
| Average optical power output per panel | 345.1 | 345.1 | 64.8 |
| (mW) | | | |
| Average power density per panel | 1.97 | 1.97 | 0.37 |
| (mW/cm²) | | | |
| Peak optical power density per panel | 2.78 | 2.78 | 0.52 |
| (mW/cm²) | | | |

### Example Device Designs

FIGS. 14-26 illustrate various example LED edge-lit device 10, according to some embodiments.

FIG. 14A-14K illustrate different views of an example edge-lit therapy device 10, according to one embodiment. In this embodiment, device 10 includes panel sections 14a and 14b housed in respective housing sections 40a and 40b pivotally connected to each other, such that device 10 may be moved between a treatment (or open) configuration and a non-treatment (or closed) configuration. Device 10 may be moved to the non-treatment (or closed) configuration for storing the device, e.g., to reduce the size of the device and/or to protect the front panel surfaces.

FIG. 14A illustrates device 10 in a treatment (or open) configuration. In this embodiment, panel sections 14a and 14b may be concave about a vertical axis A.

FIG. 14B illustrates device 10 in a non-treatment (or closed) configuration, in which panel sections 14a and 14b are folded inward toward each other.

FIG. 14C is a top view of device 10 in the non-treatment (or closed) configuration. Housing sections 40a and 40b are coupled by a connection 120, which in this embodiment comprises a hinged connection. FIG. 14D is a top view of device 10 in the treatment (or open) configuration.

FIG. 14E is a rear view of device 10 in the non-treatment (or closed) configuration. In this position, an inside edge surface 45 of each housing section 40a and 40b, along with an inside edge 24 of each panel section 14a and 14b, are visible. One or more features of device 10 may be located at the inside edge surface 45 of housing sections 40a and/or 40b. For example, in this embodiment, a port 114 for receiving a plug of an external power supply 112, and a cartridge interface 116 for receiving a time cartridge 115, are located at the inside edge surface 45 of housing section 40b. Thus, in this embodiment, the plug of external power supply 112 and cartridge 115 are received behind panel section 14b.

Further, a device configuration sensor 86 may be located at one or both inside edge surface 45, for determining whether or not device 10 is in the treatment configuration, as discussed above in the “Sensors” section. As discussed above, in some embodiments, device 10 is allowed to enter certain operational states only (e.g., a “ready” state or a “treatment” state) if it is determined to be in the open (treatment) configuration.

Because the various components discussed above are located behind the respective panel sections 14a and 14b, the panel sections 14a and 14b may extend flush with the inside edge surface 45 of housing sections 40a and 40b such that the inner edges 24 panel sections 14a and 14b may contact each other when device 10 is moved to the treatment configuration (e.g., FIG. 14A). Thus, the illuminated area presented by the front panel surface may be fully or substantially continuous across the interface between the panel sections 14a and 14b.

In some embodiments, edge reflectors 32 are applied at inner edges 24 of panel sections 14a and 14b, such that LED emitted light within each panel section 14a and 14b does not cross over into the other panel section. In other embodiments, the inner edges 24 of panel sections 14a and 14b shown in FIG. 14E are not covered by edge reflectors 32 or other opaque covering (e.g., inner edges 24 may be left bare or covered by one or more transparent layers), such that LED emitted light is permitted to travel across the interface between the inner edges 24 of panel sections 14a and 14b. Thus, LED light may pass between panel sections 14a and 14b. Such design may increase the appearance of a uniform, uninterrupted illumination across the seam between panel sections 14a and 14b.

FIG. 14F is a rear view of device 10 in the treatment (or open) configuration, illustrating example locations of hand detectors 82 and display device 100 on the rear of housing sections 40a and 40b. FIG. 14G illustrates a user 12 holding device 10 in the treatment position relative to the user's face. As shown, the user's hands are located over hand detectors 82. In this example, hand detectors 82 are generally positioned toward the lower portion of housing sections 40a and 40b (e.g., for detection of the user's hands supporting device 10 from the bottom). However, hand detectors 82 may be positioned closer to the lateral sides of housing sections 40a and 40b (e.g., for detection of the user's hands supporting device 10 from the sides), or otherwise positioned as desired.

FIGS. 14H-14K illustrate various views of device 10 indicating example locations of certain components of device 10 within housing sections 40a and 40b. In particular, FIG. 14H is a top view of device 10, FIG. 14I is a three-dimensional view generally of the front of device 10, FIG. 14J is a front view of device 10, and FIG. 14K is a side view of device 10.

As shown in FIGS. 14H-14K, housing section 40a houses a first row of LEDs 16 coupled to a heat sink 72, a panel section 14a above LEDs 16, and a first battery pack 110a and cartridge 115. In this embodiment, first battery pack 110a and cartridge 115 are positioned behind panel section 14a. Housing section 40b houses a second row of LEDs 16 coupled to heat sink 72, a panel section 14b above LEDs 16, and a second battery pack 110b, display device 100, controller 92 (e.g., housed on a printed circuit board), port 114 (for connecting to external power supply 112), and speaker 118. In this embodiment, second battery pack 110b, display device 100, controller 92, and speaker 118 are positioned behind panel section 14b.

FIG. 15A-15H illustrate different views of another example edge-lit therapy device 10, according to one embodiment. In this embodiment, device 10 includes panel sections 14a and 14b housed in respective housing sections 40a and 40b pivotally connected to each other, such that device 10 may be moved between a treatment (or open) configuration and a non-treatment (or closed) configuration. Device 10 may be moved to the non-treatment (or closed) configuration for storing the device, e.g., to reduce the size of the device and/or to protect the front panel surfaces.

FIG. 15A illustrates device 10 in a treatment (or open) configuration. In this embodiment, panel sections 14a and 14b may be concave about a vertical axis A. FIG. 15B illustrates device 10 in a non-treatment (or closed) configuration, in which panel sections 14a and 14b are folded inward toward each other.

FIG. 15C is a top view of device 10 in the non-treatment (or closed) configuration. Housing sections 40a and 40b are coupled by a connection 120, which in this embodiment comprises a hinged connection. FIG. 15D is a top view of device 10 in the treatment (or open) configuration.

FIGS. 15E-15H illustrate various views of device 10 indicating example locations of certain components of device 10 within housing sections 40a and 40b. In particular, FIG. 15H is a top view of device 10, FIG. 15I is a three-dimensional view generally of the front of device 10, FIG. 15J is a front view of device 10, and FIG. 15K is a side view of device 10.

As shown in FIGS. 15E-15H, housing section 40a houses a first row of LEDs 16 coupled to a heat sink 72, a panel section 14a above LEDs 16, a cartridge 115, and a first battery or set of batteries 110a. In this embodiment, cartridge 115 is positioned behind panel section 14a, and the first battery or set of batteries 110a is positioned beyond the left edge of panel section 14a (when device 10 is viewed from the front). Housing section 40b houses a second row of LEDs 16 coupled to heat sink 72, a panel section 14b above LEDs 16, display device 100, controller 92 (e.g., housed on a printed circuit board), port 114 (for connecting to external power supply 112), and speaker 118. In this embodiment, display device 100, controller 92, port 114, and speaker 118 are all positioned behind panel section 14b, and the second battery or set of batteries 110b is positioned beyond the right edge of panel section 14b (when device 10 is viewed from the front).

FIG. 18A-18C illustrate different views of an example embodiments of an edge-lit therapy device 10. FIG. 18A is essentially a front view of device 10 in a treatment (open) configuration. Device 10 includes a pair of panel sections 14a and 14b housed in respective housing sections 40a and 40b pivotally connected to each other, such that device 10 may be moved between a treatment (or open) configuration (FIG. 18A) and a non-treatment (or closed) configuration (FIGS. 18B and 18C). Device 10 may be moved to the non-treatment (or closed) configuration for storing the device, e.g., to reduce the size of the device and/or to protect the front panel surfaces. In this embodiment, housing sections 40a and 40b pivotally connected to each other by a pair of hinges 121. However, any number and any type(s) of hinges may be used. For example, a single hinge may extend along the full height of device 10, or two or small hinges may be spaced along the height of device 10.

As shown in FIG. 18A, each panel section 14a and 14 is concave about a vertical axis Aᵥ (or in other words, concave from side-to-side), and also concave about a horizontal axis Aₕ (or in other words, concave from top-to-bottom). In this embodiment, the degree of concavity about the vertical axis Aᵥ (side-to-side concavity) is greater than the degree of concavity about the horizontal axis Aₕ (top-to-bottom concavity). Thus, the overall shape of panel 14 may corresponds generally to a user's face, which may generally be convex from side-to-side and also convex from top-to-bottom, but to a lesser degree. Thus, for at least some users, the distance between panel 14 (including panel sections 14a and 14b) and the user's face may be relatively uniform over the treatment area of the face.

As shown in FIG. 18A, LEDs 16 are arranged in each housing section 40a and 40b along the bottom edge of the respective panel sections 14a and 14b, such that each panel section 14a and 14b is edge-lit by the respective LEDs. Also shown are four proximity detectors 80, including two chin proximity sensors 80 in the lower housing 40 and two forehead proximity sensors 80 which may be located behind panel sections 14a and 14b (or alternatively, located in the upper areas of housing sections 40a and 40b outside of the illuminating area).

FIGS. 18B and 18C illustrate two views of device 10 folded into the non-treatment (e.g. storage) configuration. In this position, particularly shown in FIG. 18C, an inside edge surface 45 of each housing section 40a and 40b, along with an inside edge 24 of each panel section 14a and 14b, are visible. One or more features of device 10 may be located at the inside edge surface 45 of housing sections 40a and/or 40b. For example, in this embodiment, a cartridge port 116 for receiving a time cartridge 115 is located at the inside edge surface 45 of housing section 40a, and a port 114 for receiving a plug of an external power supply 112 is located at the inside edge surface 45 of housing section 40b. Thus, in this embodiment, cartridge 115 and the plug of external power supply 112 are received behind panel sections 14a and 14b, respectively.

Further, a device configuration sensor 86 may be located at one or both inside edge surface 45, for determining whether or not device 10 is in the treatment configuration, as discussed above in the “Sensors” section. In this example embodiment, device configuration sensor 86 is located at the inside edge surface 45 of housing section 40b, and a corresponding conductive or reflective element 87 is located at the inside edge surface 45 of housing section 40a.

Because the various components discussed above are located behind the respective panel sections 14a and 14b, the panel sections 14a and 14b may extend flush with the inside edge surface 45 of housing sections 40a and 40b such that the inner edges 24 panel sections 14a and 14b may contact each other when device 10 is moved to the treatment configuration (see FIG. 18A). Thus, the illuminated area presented by the front panel surface may be fully or substantially continuous across the interface between the panel sections 14a and 14b.

In some embodiments, edge reflectors 32 are applied at inner edges 24 of panel sections 14a and 14b, such that LED emitted light within each panel section 14a and 14b does not cross over into the other panel section. In other embodiments, the inner edges 24 of panel sections 14a and 14b shown in FIG. 18C are not covered by edge reflectors 32 or other opaque covering (e.g., inner edges 24 may be left bare or covered by one or more transparent layers), such that LED emitted light is permitted to travel across the interface between the inner edges 24 of panel sections 14a and 14b. Thus, LED light may pass between panel sections 14a and 14b. Such design may increase the appearance of a uniform, uninterrupted illumination across the seam between panel sections 14a and 14b.

The illuminated area of panel 14 (including panel sections 14a and 14b) may have any suitable dimensions and area. In one embodiment, the illuminated area of panel 14 is about 15 cm high and about 17 cm across, with a center arc about 8 cm deep (where the 15 cm and 17 cm measurements are taken along a straight line, i.e., not following the curves of the panel), resulting in an illuminating area of about 350 cm².

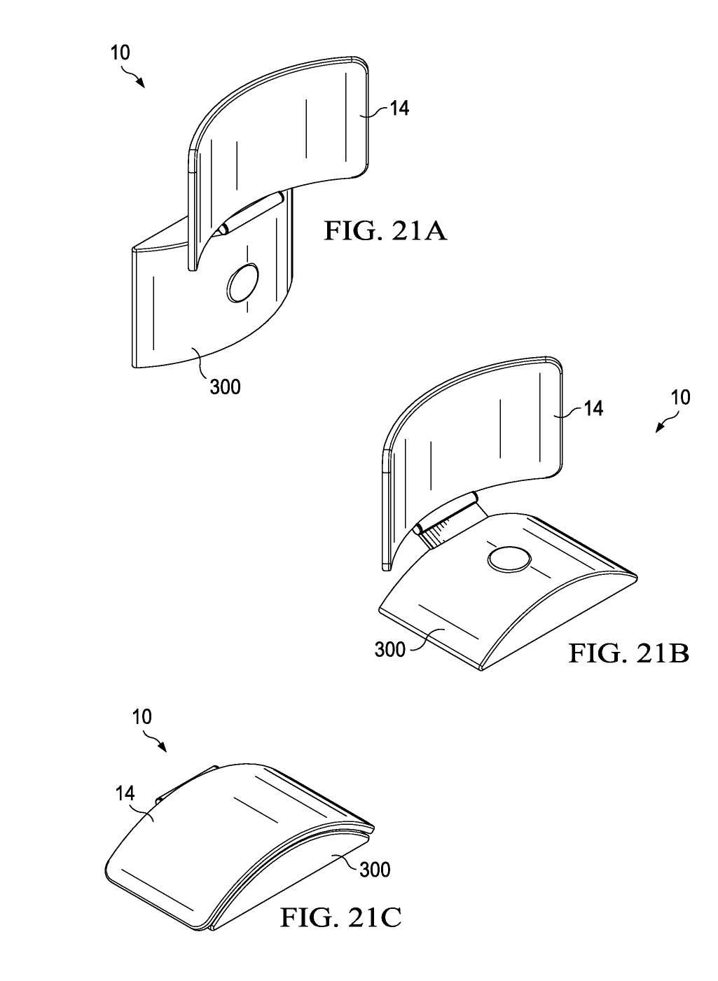

FIGS. 21-26 illustrate various other example embodiments of device 10. FIGS. 21A-21C illustrate a first treatment configuration, a second treatment configuration, and a storage configuration, respectively, of an example embodiment of an edge-lit therapy device 10 having a panel 14 foldably or pivotally attached to a base 300. As shown, panel 14 may be concave about a vertical axis.

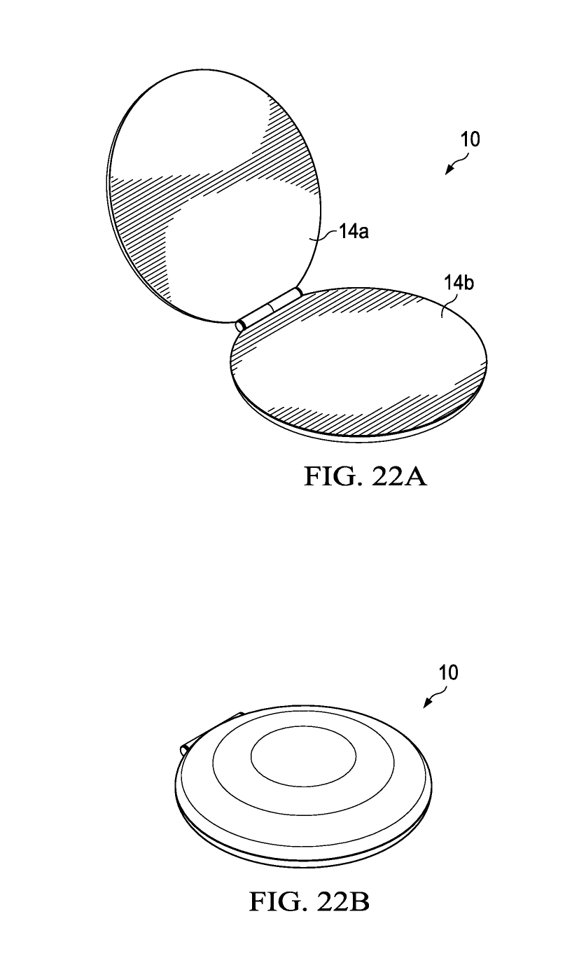

FIGS. 22A-22B illustrate a treatment configuration and a storage configuration, respectively, of an example embodiment of an edge-lit therapy device 10 having a “compact-like” shape. In this embodiment, device 10 includes two panel sections 14a and 14b connected by a hinge.

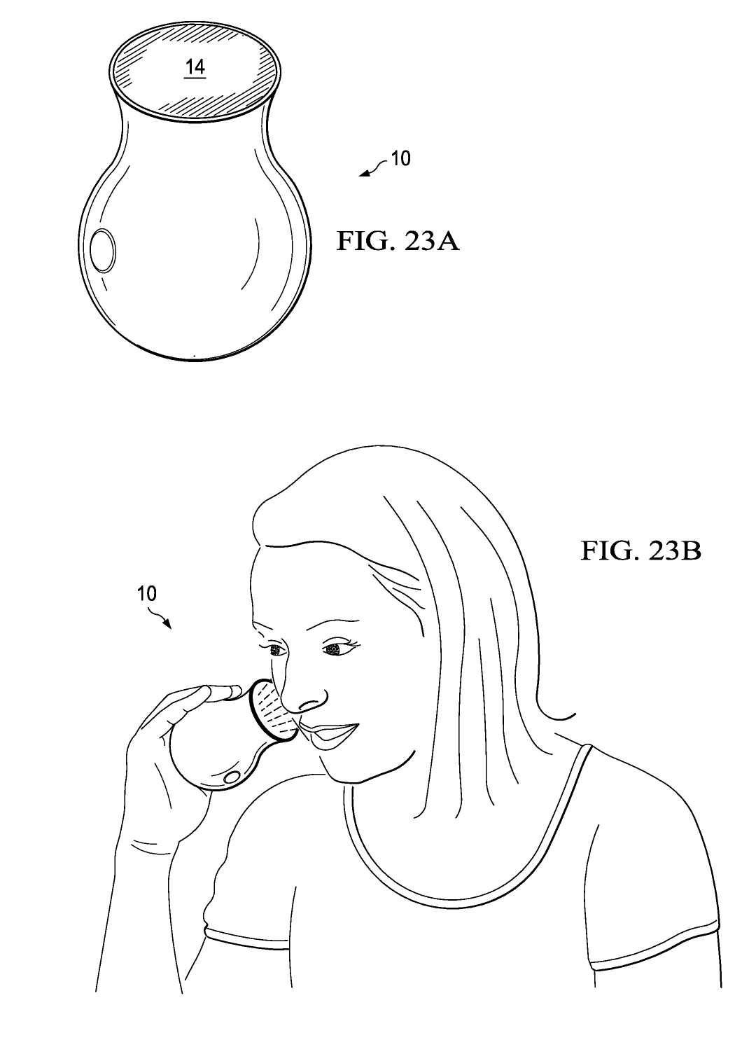

FIGS. 23A-23B illustrate an example embodiment of a relatively-small edge-lit therapy device 10 having a relatively small illuminating area. In this embodiment, the device 10 may only be able to treat one area of the face at a time. Thus, a treatment session may include a number of treatments with device 10 positioned at different locations in front of the user's face.

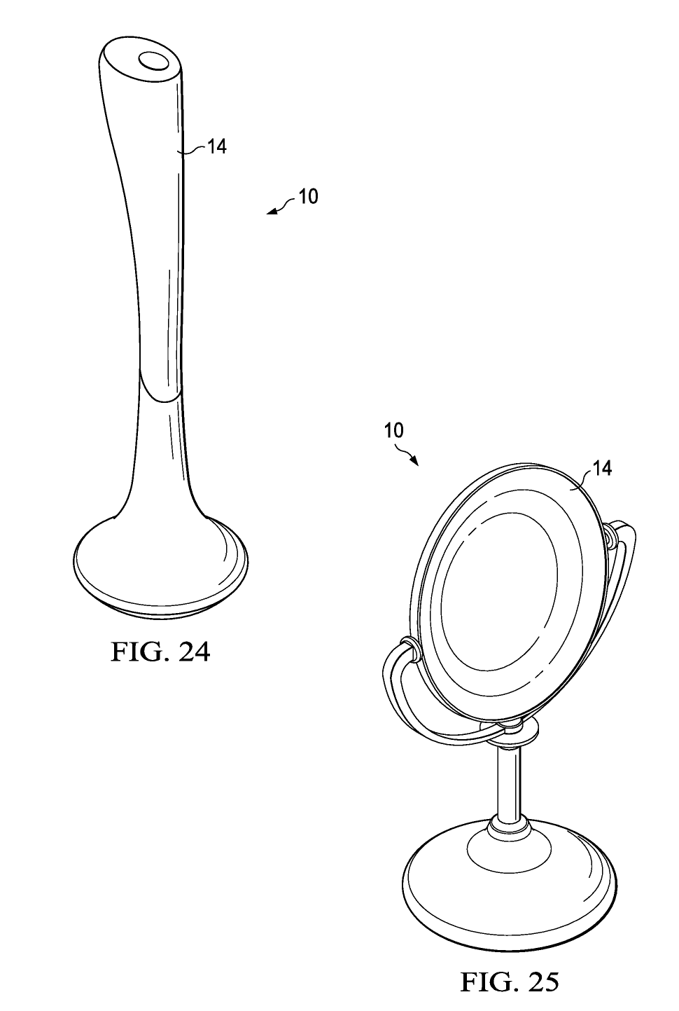

FIG. 24 illustrates an example embodiment of an edge-lit therapy device 10 having a wand-like shape. The illuminating area of panel 14 may wrap completely 360 degrees around the wand-like device, or may wrap partially around the device to any degree (e.g., 45 degrees, 60 degrees, 90 degrees, 135 degrees, 180 degrees, 270 degrees, or any other desired angle).

FIG. 25 illustrates an example embodiment of an edge-lit therapy device 10 configured similar to a vanity mirror. As shown, panel 14 may be symmetrically concave, i.e., exhibiting a spherical, parabolic, or other symmetrical bowl-like shape.

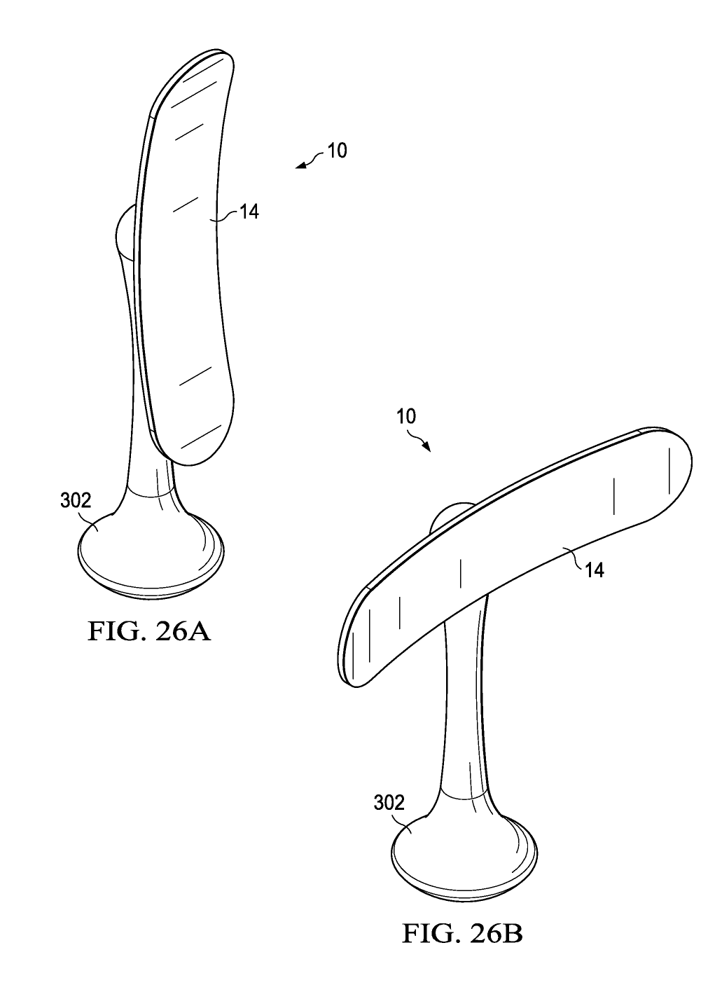

FIGS. 26A-26B illustrate an example embodiment of an edge-lit therapy device having an elongated, concave panel 14 that is rotatably mounted to a base 302. Panel 14 can thus be rotated by user 12 as desired for different types of treatments or for treating different areas of the body.

### Clinical Data

A clinical trial was performed to explore which characteristics of light exposure have the greatest influence on collagen production in fibroblasts. The following characteristics were studied: peak wavelength, peak irradiance, treatment time, treatment frequency, pulse rate (repetition rate), pulse cycle (duty cycle i.e. % of time light is on), spectral bandwidth, and adjunctive near infrared light exposure. For the study, human fibroblast cells from 4 infant foreskins grown in medium in culture plates. Arrays of light-emitting diodes were used to provide uniform illumination over the culture plates. Each plate was exposed to particular light treatment in an 8-variable 2-level experiment using partial factorial design. The study allowed the effect of low and high settings for each variable to be evaluated. Table 2 identifies the variables evaluated. In addition to the test samples, the study provided parallel samples including control samples (which had no light treatment) and benchmark samples. The benchmark samples were treated under some of the same conditions suggested by prior work to be optimal for collagen I production. See Weiss R A, Weiss M A, Geronemus R G, McDaniel D H, “A novel non-thermal non-ablative full panel LED photomodulation device for reversal of photoaging: digital microscopic and clinical results in various skin types,” 3 J Drugs Dermatol 605-610 (2004); Weiss R A, McDaniel D H, Geronemus R G, Weiss M A, “Clinical trial of a novel non-thermal LED array for reversal of photoaging: clinical, histologic, and surface profilometric results,” 36 Lasers Surg Med 85-91 (2005). The test conditions assigned to each sample are shown in Table 3.

TABLE 2

| Variable | Low setting | High setting |
|---|---|---|
| Peak wavelength | 550 nm | 590 nm |
| Peak irradiance | 6 mW/cm2 | 50 mW/cm2 |
| Treatment time | 15 sec | 180 sec |
| Treatment frequency | Alternate days | Twice daily |
| Pulse rate (repetition rate) | 3 Hz | 60 Hz |
| Pulse cycle (duty cycle, % | 10% | 71% |
| of time light is on) | | |
| Spectral bandwidth | <30 nm | >70 nm |
| Adjunctive near infrared | Off | On |
| light exposure | | |

TABLE 3

| | Variable | | | | | | | |
|---|---|---|---|---|---|---|---|---|
| | (low setting or high setting) | | | | | | | |
| | | | | Treatment | | | | |
| | Peak | Peak | Treatment | frequency | Pulse | Pulse | Spectral | |
| | wavelength | irradiance | time | (Alternate | rate | cycle | bandwidth | Adjunctive |
| | (550 nm | (6 mW/cm2 | (15 sec | days or | (3 Hz | (10% | (<30 nm | near |
| | or | or | or | twice | or | or | or | infrared^{a} |
| Sample | 590 nm) | 50 mW/cm2) | 180 sec) | daily) | 60 Hz) | 71%) | >70 nm) | (Off or on) |
| 1 | L | L | L | L | L | L | L | L |
| 2 | H | L | L | L | L | H | H | H |
| 3 | L | H | L | L | H | L | H | H |
| 4 | H | H | L | L | H | H | L | L |
| 5 | L | L | H | L | H | H | H | L |
| 6 | H | L | H | L | H | L | L | H |
| 7 | L | H | H | L | L | H | L | H |
| 8 | H | H | H | L | L | L | H | L |
| 9 | L | L | L | H | H | H | L | H |
| 10 | H | L | L | H | H | L | H | L |
| 11 | L | H | L | H | L | H | H | L |
| 12 | H | H | L | H | L | L | L | H |
| 13 | L | L | H | H | L | L | H | H |
| 14 | H | L | H | H | L | H | L | L |
| 15 | L | H | H | H | H | L | L | L |
| 16 | H | H | H | H | H | H | H | H |
| Control (1) | — | — | — | — | — | — | — | — |
| Control (2) | — | — | — | — | — | — | — | — |
| Benchmark | H | L | 35 sec | Once | L | H | 20 nm | H |
| (1) | | | | daily | | | | |
| Benchmark | H | L | 35 sec | Once | L | H | 20 nm | H |
| (2) | | | | daily | | | | |

^{a}When on, near infrared light was at 10% of peak irradiance.

In the trial, the following experimental methods were employed. Treatment order and incubation position of all samples were randomized twice daily. Half the medium was removed each day and replenished with fresh medium. At day 7, harvested cells analyzed for collagen I and glyceraldehyde 3-phosphate dehydrogenase (GAPDH) using fluorescent-label tagged immunoblotting. All collagen I results were normalized to GAPDH content, then normalized to the mean collagen I content of the control samples, and then averaged across the 4 cell lines to give one collagen value relative to control per test condition.

Collagen I content in samples under each of the 16 test conditions (normalized to collagen I content in control samples) are shown in Table 3. Some results were that collagen I production varied from ˜10% below that in control samples to ˜60% above. Certain variables alone influenced collagen I production to a level that was clearly greater than the quantitation error, including treatment time, pulse rate, and peak irradiance. For example, 15 seconds of treatment was better than 180 seconds with collagen I production increased by about 22% relative to controls. In another example, a pulse rate of 60 Hz was better than 3 Hz with collagen I production increased by about 22% relative to controls. In yet another example, a peak irradiance of 6 mW/cm² was better than 50 mW/cm² with collagen I production increased by about 22% relative to controls. When the these settings were combined (treatment of 15 seconds, pulse rate of 60 Hz, peak irradiance of 6 mW/cm²), collagen I production was a mean of 56%±16% (SE) higher than in controls. Control and benchmark conditions are means of 8 samples (2 from each of 4 cell lines). The combined conditions value is a mean of samples 9 and 10 in each cell line, so also a mean of 8 samples.

To calculate the quantitation error, the overall sample-to-sample collagen I quantitation error was estimated from 24 replicate samples. From each of the 4 cell lines, replicate samples included: one control sample, plus one benchmark sample, plus four samples randomly selected from the sixteen different test conditions.

TABLE 3

| | Collagen I |
|---|---|
| | content |
| Sample | (normalized) |
| 1 | 0.95 |
| 2 | 1.17 |
| 3 | 1.20 |
| 4 | 1.24 |
| 5 | 1.06 |
| 6 | 1.19 |
| 7 | 1.07 |
| 8 | 0.95 |
| 9 | 1.62 |
| 10 | 1.50 |
| 11 | 1.06 |
| 12 | 1.03 |
| 13 | 0.93 |
| 14 | 1.31 |
| 15 | 0.89 |
| 16 | 1.05 |

Other variables had little or no effect on collagen I production. In particular, pulse cycle had a small, or no, effect (level of collagen I production was observed to be only slightly greater than the quantitation error). Also, peak wavelength, spectral bandwidth, treatment frequency, and adjunctive near infrared exposure did not appear to influence collagen I production. The lack of increased collagen I production under benchmark conditions was unexpected and contrary to other authors' findings under similar conditions. Experimental design did not allow for complete analyses of secondary effects. However, assuming variables with largest primary effects are also responsible for secondary effects, then treatment time and pulse rate appear to be interrelated.

1. A dermatological treatment device, comprising:
a panel having internally reflective properties for reflecting light, the panel defining a front surface, a rear surface, a perimeter, and one or more edges extending along one or more portions of the perimeter;
a rear reflector adjacent the rear surface; and
one or more light emitting diodes (LEDs) positioned along at least one of the panel edges and configured to emit light into the panel such that at least a portion of the LED-emitted light reflects off the rear reflector and projects out of the panel through the panel front surface;
wherein a wavelength of the LED-emitted light is suitable for dermatological treatment, such that LED-emitted light projecting out through the front surface of the panel can be used to provide dermatological treatment to a body part positioned in front of the front surface of the panel.

2. A dermatological treatment device according to claim 1, comprising:
one or more LEDs positioned along a first panel edge; and
one or more LEDs positioned along a second panel edge generally opposite or perpendicular to the first panel edge.

3. A dermatological treatment device according to claim 1, further comprising one or more edge reflectors extending along at least one panel edge.

4. A dermatological treatment device according to claim 1, wherein:
the perimeter of the panel generally defines multiple sides;
one or more LEDs are positioned along each of one or more of the multiple sides; and
edge reflectors are positioned along each other one of the multiple sides.

5. A dermatological treatment device according to claim 1, wherein:
the panel is generally concave about a first axis and also generally concave about a second axis perpendicular to the first axis, and
the degree of concavity about the first axis is greater than the degree of concavity about the second axis.

6. A dermatological treatment device according to claim 1, wherein the one or more LEDs include different LEDs that emit different wavelengths of light.

7. A dermatological treatment device according to claim 6, wherein the one or more LEDs include at least one yellow light LED that primarily emits yellow light and at least one infrared LED that primarily emits infrared light.

8. A dermatological treatment device according to claim 6, wherein:
the perimeter of the panel generally defines multiple sides; and
different LEDs that emit different wavelengths of light are positioned along the same side of the panel.

9. A dermatological treatment device according to claim 1, wherein the rear reflector includes one or more reflector scattering features that cause diffuse reflections of the LED-emitted light.

10. A dermatological treatment device according to claim 1, wherein at least one of the panel front surface and the panel rear surface includes internal reflection frustrating features that increase the external transmission of LED-emitted light from within the panel.

11. A dermatological treatment device according to claim 10, further comprising one or more light intensity uniformity features configured to alter the relative amounts of LED-emitted light projecting out of different areas of the panel front surface, such that the intensity of light projecting from the panel is generally uniform over the area of the panel front surface, the one or more light intensity uniformity features including at least one of:
one or more reflector scattering features associated with the rear reflector;
one or more internal reflection frustrating features associated with at least one of the panel front surface and the panel rear surface; and
one or more curves in the panel.

12. A dermatological treatment device according to claim 10, further comprising one or more light intensity non-uniformity features configured to alter the relative amounts of LED-emitted light projecting out of different areas of the panel front surface, such that the intensity of light projecting through a first area of the panel front surface is greater than the intensity of light projecting through a second area of the panel front surface.

13. A dermatological treatment device according to claim 12, wherein the one or more light intensity non-uniformity features includes at least one of:
one or more reflector scattering features associated with the rear reflector;
one or more internal reflection frustrating features associated with at least one of the panel front surface and the panel rear surface; and
one or more curves in the panel.

14. A dermatological treatment device according to claim 1, further comprising one or more hand detectors configured to detect whether a user is physically holding the dermatological treatment device.

15. A dermatological treatment device according to claim 14, further comprising a controller configured to automatically switch from a first operational state and a second operational state in response to the one or more hand detectors detecting that the user is physically holding the dermatological treatment device.

16. A dermatological treatment device according to claim 1, further comprising:
one or more proximity sensors configured to determine whether a user is positioned in a treatment position relative to the dermatological treatment device; and
a controller configured to automatically switch from a first operational state and a second operational state in response to the one or more proximity sensors detecting that the user is positioned in a treatment position relative to the dermatological treatment device.

17. A dermatological treatment device for a user, comprising:
one or more hand detectors; and
one or more proximity sensors; and
a controller configured to:
receive hand detector signals from the one or more hand detectors;
determine, based on the received hand detector signals, whether the user is physically holding the dermatological treatment device,
receive proximity sensor signals from the one or more proximity sensors;
determine, based on the received proximity sensor signals, whether the user is positioned in a treatment position relative to the dermatological treatment device; and
automatically select between different states of operation based at least on the determinations of whether the user is physically holding the dermatological treatment device and whether the user is positioned in a treatment position relative to the dermatological treatment device.

18. A dermatological treatment device according to claim 17, wherein the controller is configured to:
automatically switch from a first operational state to a second operational state in response to the one or more hand detectors detecting that the user is physically holding the dermatological treatment device; and
automatically switch from the second operational state to the one or more proximity sensors detecting that the user is positioned in a treatment position relative to the dermatological treatment device.

19. A dermatological treatment device according to claim 18, wherein:
the first operational state is an “off” state;
the second operational state is a “standby” state in which the one or more LEDs are activated to emit from the panel a ready state light intensity that is lower than a treatment state light intensity; and
the third operational state is a “treatment” state in which the one or more LEDs are activated to emit the treatment state light intensity.

20. A dermatological treatment device, comprising:
a panel having internally reflective properties for reflecting light, the panel defining a front surface, a rear surface, a perimeter, and one or more edges extending along one or more portions of the perimeter;
a rear reflector adjacent the rear surface; and
a plurality of light emitting diodes (LEDs) configured to emit light into the panel such that at least a portion of the LED-emitted light projects out through the panel front surface;
wherein at least one of the LEDs emits both (a) yellow light at a first wavelength and (b) infrared light at a second wavelength different than the first wavelength; and
wherein the at least one LED is positioned along at least one of the panel edges and configured to emit light into the panel such that at least a portion of the LED-emitted light reflects off the rear reflector and projects out of the panel through the panel front surface.
